SEQUENCE LISTING (14 sequences)

SEQ ID NO: 1 - protein, 1273 aa, coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1235 aa, Artificial Sequence
  Variant SARS-CoV-2 spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGGYIPEAPRDGQAYVRKDGEWVLLSTF

SEQ ID NO: 3 - protein, 1273 aa, Artificial Sequence
  Variant SARS-CoV-2 spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 1233 aa, Artificial Sequence
  Variant SARS-CoV-2 spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGGYIPEAPRDGQAYVRKDGEWVLLSTF

SEQ ID NO: 5 - protein, 1271 aa, Artificial Sequence
  Variant SARS-CoV-2 spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 26 aa, Artificial Sequence
  T4 Fibritin trimerization domain
GYIPEAPRDGQAYVRKDGEWVLLSTF

SEQ ID NO: 7 - protein, 173 aa, Artificial Sequence
  protein nanoparticle subunit
ESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 8 - protein, 154 aa, Artificial Sequence
  protein nanoparticle subunit
MQIYEGKLTAEGLRFGIVASRFNHALVDRLVEGAIDAIVRHGGREEDITLVRVPGSWEIPVAAGELARKEDIDAVIAIGVLIRGATPHFDYIASEVSKGLADLSLELRKPITFGVITADTLEQAIERAGTKHGNKGWEAALSAIEMANLFKSLR

SEQ ID NO: 9 - protein, 265 aa, Artificial Sequence [sequence omitted]

SEQ ID NO: 10 - protein, 265 aa, Artificial Sequence [sequence omitted]

SEQ ID NO: 11 - DNA, 3822 bp, coronavirus
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 12 - protein, 226 aa, Artificial Sequence
  protein nanoparticle subunit
MDSKGSSQKGSRLLLLLVVSNLLLPQGVVGAHIVMVDAYKPTKGSGSAMQIYEGKLTAEGLRFGIVASRFNHALVDRLVEGAIDAIVRHGGREEDITLVRVPGSWEIPVAAGELARKENISAVIAIGVLIRGATPHFDYIASEVSKGLADLSLELRKPITFGVITADTLEQAIERAGTKHGNKGWEAALSAIEMANLFKSLRGGLVPRGSHHHHHHSAWSHPQFEK

SEQ ID NO: 13 - protein, 244 aa, Artificial Sequence
  protein nanoparticle subunit
MDSKGSSQKGSRLLLLLVVSNLLLPQGVVGQHHHHHHHHSAWSHPQFEKGGLVPRGGAHIVMVDAYKPTKGGGSGDPMLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQNISESINNIVDHAIKSKDHATFNFLQWYVAEQHEEEVLFKDILDKIELIGNENHGLYLADQYVKGIAKSRKS

SEQ ID NO: 14 - protein, 1820 aa, Artificial Sequence
  Variant SARS-CoV-2 spike protein
MGWSCIILFLVATATGVHSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGKGGSGGGGSGGLEVLFQGPQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSGGGLVPQQSGDSATHIKFSKRDEDGKELAGATMELRDSSGKTISTWISDGQVKDFYLYPGKYTFVETAAPDGYEVATAITFTVNEQGQVTVNGKATKGDAHI

(19) United States
(12) Patent Application Publication
(10) Pub. No.: US 2023/0113170 A1
(43) Pub. Date: Apr. 13, 2023

(54) SARS-COV-2 VACCINE

(71) Applicants: The United States of America, as represented by the Secretary, Department of Health and Human, Bethesda, MD (US); Board of Regents, The University of Texas System, Austin, TX (US); Trustees of Dartmouth College, Hanover, NH (US)

(72) Inventors: Barney Graham, Smyma, GA (US); Kizzmekia Corbett, Bethesda, MD (US); Olubukola Abiona, University Park, MD (US); Geoffrey Hutchinson, Rockville, MD (US); Jason McLellan, Austin, TX (US); Daniel Wrapp, Austin, TX (US); Nianshuang Wang, Austin, TX (US)

(73) Assignees: The United States of America, as represented by the Secretary, Department of Health and Human, Bethesda, MD (US); Board of Regents, The University of Texas System, Austin, TX (US); Trustees of Dartmouth College, Hanover, NH (US)

(21) Appl. No.: 17/798,021

(22) PCT Filed: Feb. 11, 2021
(86) PCT No.: PCT/US2021/017709

§ 371 Date: Aug. 5, 2022

Related U.S. Application Data
(60) Provisional application No. 62/972,886, filed on Feb. 11, 2020.

(51) Int. Cl. A61K 39/215; C12N 15/86; A61P 31/14

(52) U.S. Cl. CPC A61K 39/215; C12N 15/86; A61P 31/14; C12N 2770/20034; A61K 39/39

(57) ABSTRACT

SARS-CoV-2 S ectodomain trimers stabilized in a prefusion conformation, nucleic acid molecules and vectors encoding these proteins, and methods of their use and production are disclosed. In several embodiments, the SARS-CoV-2 S ectodomain trimers and/or nucleic acid molecules can be used to generate an immune response to SARS-CoV-2 S in a subject, for example, an immune response that inhibits SARS-CoV-2 infection in the subject.

### CROSS REFERENCE TO RELATED APPLICATION

This application claims the benefit of U.S. Provisional Application No. 62/972,886, filed Feb. 11, 2020, which is incorporated by reference herein in its entirety.

### FIELD

This disclosure relates to recombinant SARS-CoV-2 spike (S) protein that is stabilized in a prefusion conformation, and its use as an immunogen.

### BACKGROUND

Coronaviruses are enveloped, positive-sense single-stranded RNA viruses. They have the largest genomes (26-32 kb) among known RNA viruses, and are phylogenetically divided into four genera (α, β, γ, δ), with betacoronaviruses further subdivided into four lineages (A, B, C, D). Coronaviruses infect a wide range of avian and mammalian species, including humans.

In 2019, a novel coronavirus (designated SARS-CoV-2 by the World Health Organization) was identified as the causative agent of a coronavirus pandemic that appears to have originated in Wuhan, China. The high case-fatality rate, vaguely defined epidemiology, and absence of prophylactic or therapeutic measures against coronaviruses have created an urgent need for an effective vaccine and related therapeutic agents.

### SUMMARY

Disclosed herein are recombinant SARS-CoV-2 S ectodomain trimers comprising protomers comprising one or more amino acid substitutions that stabilize the S protein trimer in the prefusion conformation.

In some embodiments, the recombinant SARS-CoV-2 S ectodomain trimer comprises protomers comprising an amino acid sequence at least 95% (such as at least 96%, at least 97%, at least 98%, or at least 99%) identical to residues 16-1208 of SEQ ID NO: 2 and proline substitutions at positions 986 and 987 of SEQ ID NO: 2, wherein the prolines stabilize the S ectodomain trimer in a prefusion conformation. The prolines at positions 986 and 987 are amino acid substitutions compared to native SARS-CoV-2 S ectodomain sequence, such as K986P and V987P substitutions. In some embodiments, the recombinant SARS-CoV-2 S ectodomain trimer comprises protomers comprising residues 16-1208 of SEQ ID NO: 2.

In some embodiments, the protomers of the recombinant SARS-CoV-2 S ectodomain trimer further comprise one or more additional amino acid substitutions or deletions, such as amino acid substitutions that stabilize the recombinant SARS-CoV-2 S ectodomain trimer in the prefusion conformation, or amino acid substitutions to inhibit or prevent protease cleavage at a S1/S2 protease cleavage site of the S ectodomain.

In some embodiments, the protomers of the recombinant SARS-CoV-2 S ectodomain trimer can be linked to a trimerization domain (such as T4 Fibritin trimerization domain). In additional embodiments, the protomers of the recombinant SARS-CoV-2 S ectodomain trimer can be membrane anchored, for example, by linkage to a transmembrane domain.

In additional embodiments, the recombinant SARS-CoV-2 S ectodomain trimer can be included on a self-assembling protein nanoparticle, such as a ferritin protein nanoparticle, or a synthetic protein-based nanoparticle. Nucleic acid molecules encoding a protomer of the disclosed recombinant SARS-CoV-2 S ectodomain trimers are also provided, as are vectors including the nucleic acid molecules, and methods of producing the disclosed recombinant SARS-CoV-2 S ectodomain trimers.

Immunogenic compositions including the recombinant SARS-CoV-2 S ectodomain trimer that are suitable for administration to a subject are also provided, and may also be contained in a unit dosage form. The compositions can further include an adjuvant. The recombinant SARS-CoV-2 S ectodomain trimers may also be conjugated to a carrier to facilitate presentation to the immune system. Methods of inducing an immune response in a subject are disclosed, as are methods of inhibiting or preventing SARS-CoV-2 infection in a subject, by administering to the subject an effective amount of a disclosed recombinant SARS-CoV-2 S ectodomain trimer, nucleic acid molecule, or vector.

The foregoing and other features and advantages of this disclosure will become more apparent from the following detailed description of several embodiments which proceeds with reference to the accompanying figures.

### BRIEF DESCRIPTION OF THE FIGURES

FIGS. 1A-1D. Stabilization of SARS-CoV-2 S protein in a prefusion conformation by K986P and V987P amino acid substitutions. (FIG. 1A) Schematic of SARS-CoV-2 S primary structure. SS=signal sequence, NTD=N-terminal domain, RBD=receptor-binding domain, S1/S2=S1/S2 protease cleavage site, FP=fusion peptide, HR1=heptad repeat 1, CH=central helix, CD=connector domain, HR2=heptad repeat 2, TM=transmembrane domain, CT=cytoplasmic tail. Arrow denotes protease cleavage site. (FIG. 1B) Size exclusion chromatography of SARS-CoV-2 S-2P protein (SEQ ID NO: 2) resulted in single large peak demonstrating high levels of protein expression from a DNA plasmid and a uniform population of protein. Peak fractions eluted as expected based on protein size. 2D class averages (FIG. 1C) and 4.7 Angstrom structure (FIG. 1D) of SARS-CoV-2 spike proteins reveal solely prefusion conformation.

FIGS. 2A-2D. Antibody responses in multiple mouse strains following immunization with SARS-CoV-2 WT or S-2P. BALB/cJ (FIG. 2A, 2D), C57BL/6J (FIG. 2B), or B6C3F1/J (FIG. 2C) mice were immunized at weeks 0 and 3 with PBS, 0.01 μg, 0.1 μg, or 1 μg of SARS-CoV-2 S WT or SARS-CoV-2 S-2P adjuvanted with Sigma Adjuvant System (SAS), and sera were collected 2 weeks post-prime (unfilled circles) and 2 weeks post-boost (filled circles). Sera from SARS-CoV-2 S-2P immunized mice were assessed for SARS-CoV-2 S-specific IgG by ELISA (FIGS. 2A-2C). Post-boost sera from both S WT and S-2P-immunized BALB/cJ mice were assessed for neutralizing antibodies against homotypic SARS-CoV-2 pseudovirus (FIG. 2D). Two-way ANOVA with multiple comparisons tests was used to compare post-prime and post-boost binding antibody responses within each dose level and between doses post-boost (FIGS. 2A-2C) and to compare neutralizing antibodies elicited by S WT vs. S-2P at each dose and effects of dose on neutralizing activity (FIG. 2D). Dotted line represents assay limit of detection. gray dashed line=p-value <0.05, gray line=p-value <0.01, black dashed line=p-value <0.001, black line=p-value <0.0001.

FIGS. 3A-3B. Ability of SARS-CoV-2 S WT and SARS-CoV-2 S-2P to protect mice against viral replication. BALB/cJ mice were immunized at weeks 0 and 3 with PBS, 0.01 μg, 0.1 μg, or 1 μg of SARS-CoV-2 S WT or SARS-CoV-2 S-2P adjuvanted with SAS. Four weeks post-boost, mice were challenged with mouse-adapted SARS-CoV-2. Two days post-challenge, at peak viral load, lungs (FIG. 3A) and nasal turbinates (FIG. 3B) were harvested for assessment of viral load by plaque assay. Groups were compared by one-way AVOVA with multiple comparisons test. Dotted line represents assay limit of detection. gray dashed line=p-value <0.05, gray line=p-value <0.01. Note: 0.01 μg S-2P-immunized mice were not challenged (N/T), due to death unrelated to the experiment.

FIGS. 4A-4E. Lumazine Synthase (LuS)- and ferritin-nanoparticle scaffolds with N-linked glycan and bioconjugation tag (SpyTag) express well as assembled nanoparticles in mammalian cells. (FIG. 4A) Schematic diagram showing the separate CnaB2 domain tag (“SpyTag”) and remaining CnaB2 domain (“SpyCatcher”) for bioconjugation through an isopeptide bond as a means to covalently link molecules via the SpyTag and SpyCatcher bioconjugation pair. (FIG. 4B) Design of expression constructs to produce activated nanoparticles with SpyTag in mammalian cells for conjugating antigens on the nanoparticle surface. Upper panel shows the DNA construct. A SpyTag was placed at the N-terminus of the nanoparticle sequence after the cleavable signal peptide. His and Strep tags were placed at the C-terminus of the LuS nanoparticle. An N-linked glycosylation site was engineered in the nanoparticle sequence to facilitate protein expression. Lower panels show the expected structures of the LuS-N71-SpyTag and ferritin-N96-SpyTag monomers and assembled nanoparticles. Both glycan and SpyTag are on the nanoparticle surface. (FIG. 4C) Size exclusion chromatograms confirmed the correct sizes of the nanoparticles. The samples were loaded on a Superdex 200 Increase 10/300 GL column in PBS. (FIG. 4D) SDS-PAGE of LuS-N71-SpyTag and ferritin-N96-SpyTag in the presence or absence of PNGase F. The position of PNGase F is marked. The multiple bands for ferritin are likely due to proteolytic cleavage and incomplete glycosylation. (FIG. 4E) Negative stain EM images (left panels) and 2D class averages (right panels) of LuS-N71-SpyTag and ferritin-N96-SpyTag show the correct assembly of the purified nanoparticles with expected sizes.

FIGS. 5A-5E. Conjugation of SARS-CoV-2 S trimer to LuS-SpyTag displays SARS-CoV-2 spike trimer on the surface of the LuS-N71-SpyLinked-CoV-2 spike nanoparticle. (FIG. 5A) Schematic diagram showing conjugation of SpyTag-coupled LuS to SpyCatcher-coupled SARS-CoV-2 spike trimer to make LuS-N71-SpyLinked-CoV-2 spike nanoparticle. (FIG. 5B) SEC profiles of LuS-N71-SpyTag, SARS-CoV-2 spike-SpyCatcher, and the conjugated product LuS-N71-SpyLinked-CoV-2 spike on a Superdex 200 Increase 10/300 GL column in PBS. (FIG. 5C) SDS-PAGE of LuS-N71-SpyTag (lane 1), SARS-CoV-2 spike-SpyCatcher (lane 2), and the conjugation mixture of LuS-N71-SpyTag with SARS-CoV-2 spike-SpyCatcher (lane 3) in the presence of DTT. The conjugation mixture (lane 3) shows the conjugated LuS-N71-SpyLinked-CoV-2 spike nanoparticle with minor excess of LuS-N71-SpyTag. (FIG. 5D) Negative stain EM of the LuS-N71-SpyLinked-CoV-2 spike nanoparticle after SEC purification showing representative micrographs (left panel) and 2D class average (right panel). (FIG. 5E) Surface Plasmon Resonance (SPR) response curves for LuS-N71-SpyLinked-CoV-2 spike nanoparticle binding with RBD-targeting antibody CR3022 IgG, with IgG coupled to chip and nanoparticle in solution. A series of nanoparticle concentrations was analyzed in which the concentration of SARS CoV-2 spike coupled to the nanoparticle ranged from 200 nM to 1.56 nM. Observed kₐ value provided.

FIGS. 6A-6C. Immunogenicity of LuS-N71-SpyLinked-CoV-2 spike. (FIG. 6A) Schematic immunization procedures for SARS-CoV-2 spike immunogens. (FIG. 6B) Serum assessment of anti-SARS-CoV-2 spike ELISA titers. Immunization groups are color-coded. Vertical dotted lines separate immunogen dose groups and weeks post prime. Starting reciprocal serum dilution (100) is indicated with a horizontal dashed line. ELISA titer from each animal is shown as an individual dot. Triangle-shape dot provided for ELISA titers at assay maximum. Geometric means indicated by black horizontal lines. Note that the three animals immunized with 0.08 mg LuS-N71-SpyTag, which showed high ELISA titers at week 5, were the same three animals of this control group that showed detectable neutralization. (FIG. 6C) Neutralization titer from each animal at week 5 is shown as an individual dot, and geometric means are indicated by black horizontal lines with values provided for each group. Immunization groups are color-coded as in FIG. 6B. Limit of detection (titer=40) indicated with a horizontal dashed line. P values determined by two-tailed Mann-Whitney tests. * indicates P≤0.05, ** indicates P≤0.01, *** indicates P≤0.001 and **** indicates P≤0.0001.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file in the form of the file named “Sequence.txt” (˜88 kb), which was created on Feb. 11, 2021, which is incorporated by reference herein.

### DETAILED DESCRIPTION

This disclosure provides SARS-CoV-2 Spike glycoprotein (S) ectodomain trimers that are stabilized in the prefusion conformation and which are useful, for example, to elicit a neutralizing immune response in a subject.

### I. SUMMARY OF TERMS

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

Adjuvant: A vehicle used to enhance antigenicity. In some embodiments, an adjuvant can include a suspension of minerals (alum, aluminum hydroxide, or phosphate) on which antigen is adsorbed; or water-in-oil emulsion, for example, in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). In some embodiments, the adjuvant used in a disclosed immunogenic composition is a combination of lecithin and carbomer homopolymer (such as the ADJUPLEX™ adjuvant available from Advanced BioAdjuvants, LLC, see also Wegmann, Clin Vaccine Immunol, 22(9): 1004-1012, 2015). Additional adjuvants for use in the disclosed immunogenic compositions include the QS21 purified plant extract, Matrix M, AS01, MF59, and ALFQ adjuvants. Immunostimulatory oligonucleotides (such as those including a CpG motif) can also be used as adjuvants. Adjuvants include biological molecules (a “biological adjuvant”), such as costimulatory molecules. Exemplary adjuvants include IL-2, RANTES, GM-CSF, TNF-α, IFN-γ, G-CSF, LFA-3, CD72, B7-1, B7-2, OX-40L, 4-1BBL and toll-like receptor (TLR) agonists, such as TLR-9 agonists. Additional description of adjuvants can be found, for example, in Singh (ed.) Vaccine Adjuvants and Delivery Systems. Wiley-Interscience, 2007). Adjuvants can be used in combination with the disclosed immunogens.

Administration: The introduction of an agent, such as a disclosed immunogen, into a subject by a chosen route. Administration can be local or systemic. For example, if the chosen route is intranasal, the agent (such as an immunogen comprising a recombinant SARS-CoV-2 S ectodomain trimer stabilized in a prefusion conformation) is administered by introducing the composition into the nasal passages of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Amino acid substitution: The replacement of one amino acid in a polypeptide with a different amino acid.

Antibody: An immunoglobulin, antigen-binding fragment, or derivative thereof, that specifically binds and recognizes an analyte (antigen) such as a SARS-CoV-2 S protein, an antigenic fragment thereof, or a dimer or multimer of the antigen. The term “antibody” is used herein in the broadest sense and encompasses various antibody structures, including but not limited to monoclonal antibodies, polyclonal antibodies, multispecific antibodies (e.g., bispecific antibodies), and antibody fragments, so long as they exhibit the desired antigen-binding activity. Non-limiting examples of antibodies include, for example, intact immunoglobulins and variants and fragments thereof that retain binding affinity for the antigen. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies (see, e.g., Kontermann and Dubel (Ed), Antibody Engineering, Vols. 1-2, 2^{nd} Ed., Springer Press, 2010).

Carrier: An immunogenic molecule to which an antigen can be linked. When linked to a carrier, the antigen may become more immunogenic. Carriers are chosen to increase the immunogenicity of the antigen and/or to elicit antibodies against the carrier which are diagnostically, analytically, and/or therapeutically beneficial. Useful carriers include polymeric carriers, which can be natural (for example, proteins from bacteria or viruses), semi-synthetic or synthetic materials containing one or more functional groups to which a reactant moiety can be attached.

Conservative variants: “Conservative” amino acid substitutions are those substitutions that do not substantially affect or decrease a function of a protein, such as the ability of the protein to induce an immune response when administered to a subject. The term conservative variation also includes the use of a substituted amino acid in place of an unsubstituted parent amino acid. Furthermore, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

The following six groups are examples of amino acids that are considered to be conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

Non-conservative substitutions are those that reduce an activity or function of the recombinant SARS-CoV-2 S ectodomain trimer, such as the ability to induce an immune response when administered to a subject. For instance, if an amino acid residue is essential for a function of the protein, even an otherwise conservative substitution may disrupt that activity. Thus, a conservative substitution does not alter the basic function of a protein of interest.

Control: A reference standard. In some embodiments, the control is a negative control sample obtained from a healthy patient. In other embodiments, the control is a positive control sample obtained from a patient diagnosed with a SARS-CoV-2 infection, such as SARS-CoV-2. In still other embodiments, the control is a historical control or standard reference value or range of values (such as a previously tested control sample, such as a group of patients infected with a SARS-CoV-2 with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Coronavirus: A family of positive-sense, single-stranded RNA viruses that are known to cause severe respiratory illness. Viruses currently known to infect humans from the coronavirus family are from the alphacoronavirus and betacoronavirus genera. Additionally, it is believed that the gammacoronavirus and deltacoronavirus genera may infect humans in the future.

Non-limiting examples of betacoronaviruses include SARS-CoV-2, Middle East respiratory syndrome coronavirus (MERS-CoV), Severe Acute Respiratory Syndrome coronavirus (SARS-CoV), Human coronavirus HKU1 (HKU1-CoV), Human coronavirus OC43 (OC43-CoV), Murine Hepatitis Virus (MHV-CoV), Bat SARS-like coronavirus WIV1 (WIV1-CoV), and Human coronavirus HKU9 (HKU9-CoV). Non-limiting examples of alphacoronaviruses include human coronavirus 229E (229E-CoV), human coronavirus NL63 (NL63-CoV), porcine epidemic diarrhea virus (PEDV), and Transmissible gastroenteritis coronavirus (TGEV). A non-limiting example of a deltacoronavirus is the Swine Delta Coronavirus (SDCV).

The viral genome is capped, polyadenylated, and covered with nucleocapsid proteins. The coronavirus virion includes a viral envelope containing type I fusion glycoproteins referred to as the spike (S) protein. Most coronaviruses have a common genome organization with the replicase gene included in the 5′-portion of the genome, and structural genes included in the 3′-portion of the genome.

Degenerate variant: In the context of the present disclosure, a “degenerate variant” refers to a polynucleotide encoding a polypeptide that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

Effective amount: An amount of agent, such as an immunogen, that is sufficient to elicit a desired response, such as an immune response in a subject. It is understood that multiple administrations of a disclosed immunogen may be needed to obtain a protective immune response against an antigen of interest, and/or administration of a disclosed immunogen as the “prime” in a prime boost protocol wherein the boost immunogen can be different from the prime immunogen. Accordingly, an effective amount of a disclosed immunogen can be the amount of the immunogen sufficient to elicit a priming immune response in a subject that can be subsequently boosted with the same or a different immunogen to elicit a protective immune response.

In one example, a desired response is to inhibit or reduce or prevent SARS-CoV-2 infection. The SARS-CoV-2 infection does not need to be completely eliminated or reduced or prevented for the method to be effective. For example, administration of an effective amount of the immunogen can induce an immune response that decreases the SARS-CoV-2 infection (for example, as measured by infection of cells, or by number or percentage of subjects infected by the SARS-CoV-2) by a desired amount, for example by at least 50%, at least 60%, at least 70%, at least 80%, at least 90%, at least 95%, at least 98%, or even at least 100% (elimination or prevention of detectable SARS-CoV-2 infection), as compared to a suitable control.

Epitope: An antigenic determinant. These are particular chemical groups or peptide sequences on a molecule that are antigenic, such that they elicit a specific immune response, for example, an epitope is the region of an antigen to which B and/or T cells respond. An antibody can bind to a particular antigenic epitope, such as an epitope on SARS-CoV-2 S ectodomain. Epitopes can be formed both from contiguous amino acids or noncontiguous amino acids juxtaposed by tertiary folding of a protein.

Expression: Transcription or translation of a nucleic acid sequence. For example, a gene is expressed when its DNA is transcribed into an RNA or RNA fragment, which in some examples is processed to become mRNA. A gene may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. The term “expression” is used herein to denote either transcription or translation. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included. For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

Ferritin: A protein that stores iron and releases it in a controlled fashion. The protein is produced by almost all living organisms. Ferritin polypeptides assemble into a globular protein complex of 24 protein subunits, and each of the 24 subunits includes a single ferritin polypeptide. In some examples, ferritin is used to form a nanoparticle presenting antigens on its surface, for example, a SARS-CoV-2 S ectodomain trimer.

Heterologous: Originating from a different genetic source. A nucleic acid molecule that is heterologous to a cell originated from a genetic source other than the cell in which it is expressed. In one specific, non-limiting example, a heterologous nucleic acid molecule encoding a recombinant SARS-CoV-2 S ectodomain is expressed in a cell, such as a mammalian cell. Methods for introducing a heterologous nucleic acid molecule in a cell or organism are well known in the art, for example injection of a nanoparticle containing a nucleic acid encoding a disclosed immunogen, or transformation with the nucleic acid, including electroporation, lipofection, particle gun acceleration, and homologous recombination.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Immune response: A response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies.

Immunogen: A compound, composition, or substance (for example, a recombinant SARS-CoV-2 S ectodomain trimer) that can elicit an immune response in an animal, including compositions that are injected or absorbed into an animal. Administration of an immunogen to a subject can lead to protective immunity against a pathogen of interest.

Immunogenic composition: A composition comprising a disclosed recombinant SARS-CoV-2 S ectodomain trimer that induces a measurable CTL response against the SARS-CoV-2, or induces a measurable B cell response (such as production of antibodies) against the SARS-CoV-2, when administered to a subject. It further refers to isolated nucleic acid molecules and vectors encoding a protomer of a disclosed recombinant SARS-CoV-2 S ectodomain trimer that can be used to express the protomer (and thus be used to elicit an immune response against recombinant SARS-CoV-2 S ectodomain trimer). For in vivo use, the immunogenic composition will typically include the recombinant SARS-CoV-2 S ectodomain trimer or a nucleic acid molecule encoding a protomer of the recombinant SARS-CoV-2 S ectodomain trimer in a pharmaceutically acceptable carrier and may also include other agents, such as an adjuvant.

Inhibiting or treating a disease: Inhibiting the full development of a disease or condition, for example, in a subject who is at risk for a disease such as a SARS-CoV-2 infection. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

Isolated: An “isolated” biological component has been substantially separated or purified away from other biological components, such as other biological components in which the component naturally occurs, such as other chromosomal and extrachromosomal DNA, RNA, and proteins. Proteins, peptides, nucleic acids, and viruses that have been “isolated” include those purified by standard purification methods. Isolated does not require absolute purity, and can include protein, peptide, nucleic acid, or virus molecules that are at least 50% isolated, such as at least 75%, 80%, 90%, 95%, 98%, 99%, or even 99.9% isolated.

Linker and Linked: A bi-functional molecule that can be used to link two molecules into one contiguous molecule. Non-limiting examples of peptide linkers include glycine-serine peptide linkers. Unless context indicates otherwise, reference to “linking” a first polypeptide and a second polypeptide, or to two polypeptides “linked” together, or to a first polypeptide having a “linkage” to a second polypeptide, refers to covalent linkage (for example via a peptide linker such that the first and second polypeptides form a contiguous polypeptide chain). If a peptide linker is involved, the covalent linkage of the first and second polypeptides can be to the N- and C-termini of the peptide linker. Typically, such linkage is accomplished using molecular biology techniques to genetically manipulate DNA encoding the first polypeptide linked to the second polypeptide by the peptide linker.

Native protein, sequence, or disulfide bond: A polypeptide, sequence or disulfide bond that has not been modified, for example, by selective mutation. For example, selective mutation to focus the antigenicity of the antigen to a target epitope, or to introduce a disulfide bond into a protein that does not occur in the native protein. Native protein or native sequence are also referred to as wild-type protein or wild-type sequence. A non-native disulfide bond is a disulfide bond that is not present in a native protein, for example, a disulfide bond that forms in a protein due to introduction of one or more cysteine residues into the protein by genetic engineering.

Nucleic acid molecule: A polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein-coding regions, in the same reading frame.

Pharmaceutically acceptable carriers: The pharmaceutically acceptable carriers of use are conventional. Remington's Pharmaceutical Sciences, by E. W. Martin, Mack Publishing Co., Easton, Pa., 19th Edition, 1995, describes compositions and formulations suitable for pharmaceutical delivery of the disclosed immunogens.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions (such as immunogenic compositions) to be administered can contain minor amounts of non-toxic auxiliary substances, such as wetting or emulsifying agents, preservatives, and pH buffering agents and the like, for example sodium acetate or sorbitan monolaurate. In particular embodiments, suitable for administration to a subject the carrier may be sterile, and/or suspended or otherwise contained in a unit dosage form containing one or more measured doses of the composition suitable to induce the desired immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Polypeptide: Any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

Prime-boost vaccination: An immunotherapy including administration of a first immunogenic composition (the prime vaccine) followed by administration of a second immunogenic composition (the boost vaccine) to a subject to induce an immune response. In some examples, the prime vaccine and/or the boost vaccine include a vector (such as a viral vector, RNA, or DNA vector) expressing the antigen to which the immune response is directed. The boost vaccine is administered to the subject after a suitable time interval from administration of the prime vaccine, and examples of such timeframes are disclosed herein. In some embodiments, the prime vaccine, the boost vaccine, or both, additionally include an adjuvant. In one non-limiting example, the prime vaccine is a DNA-based vaccine (or other vaccine based on gene delivery), and the boost vaccine is a protein subunit or protein nanoparticle based vaccine.

Protein nanoparticle: A multi-subunit, self-assembling, protein-based polyhedron shaped structure. The subunits are each composed of proteins (for example a glycosylated polypeptide), and, optionally of single or multiple features of the following: nucleic acids, prosthetic groups, organic and inorganic compounds. In some embodiments, protomers of the disclosed trimeric spike proteins can be fused or conjugated to the subunits of the protein nanoparticles to provide multiple copies of the trimeric spike on each protein nanoparticle. Non-limiting examples of protein nanoparticles include ferritin nanoparticles (see, e.g., Zhang, Y. Int. J. Mol. Sci., 12:5406-5421, 2011, incorporated by reference herein), encapsulin nanoparticles (see, e.g., Sutter et al., Nature Struct. and Mol. Biol., 15:939-947, 2008, incorporated by reference herein), Sulfur Oxygenase Reductase (SOR) nanoparticles (see, e.g., Urich et al., Science, 311:996-1000, 2006, incorporated by reference herein), lumazine synthase nanoparticles (see, e.g., Zhang et al., J. Mol. Biol., 306: 1099-1114, 2001), and pyruvate dehydrogenase nanoparticles (see, e.g., Izard et al., PNAS 96: 1240-1245, 1999, incorporated by reference herein). Ferritin, encapsulin, SOR, lumazine synthase, and pyruvate dehydrogenase are monomeric proteins that self-assemble into a globular protein complexes that in some cases consists of 24, 60, 24, 60, and 60 protein subunits, respectively. Additional protein nanoparticle structures are described by Heinze et al., J Phys Chem B., 120(26):5945-52, 2016; Hsia et al., Nature, 535(7610):136-9, 2016; and King et al., Nature, 510(7503):103-8, 2014; each of which is incorporated by reference herein.

Recombinant: A recombinant nucleic acid molecule is one that has a sequence that is not naturally occurring, for example, includes one or more nucleic acid substitutions, deletions or insertions, and/or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques. A recombinant virus is one that includes a genome that includes a recombinant nucleic acid molecule. A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell, or into the genome of a recombinant virus.

SARS-CoV-2: Also known as Wuhan coronavirus, 2019-nCoV, or 2019 novel coronavirus, SARS-CoV-2 is a positive-sense, single stranded RNA virus of the genus betacoronavirus that has emerged as a highly fatal cause of severe acute respiratory infection. The viral genome is capped, polyadenylated, and covered with nucleocapsid proteins. The SARS-CoV-2 virion includes a viral envelope with large spike glycoproteins. The SARS-CoV-2 genome, like most coronaviruses, has a common genome organization with the replicase gene included in the 5′-two thirds of the genome, and structural genes included in the 3′-third of the genome. The SARS-CoV-2 genome encodes the canonical set of structural protein genes in the order 5′-spike (S)—envelope (E)—membrane (M) and nucleocapsid (N)—3′. Symptoms of SARS-CoV-2 infection include fever and respiratory illness, such as dry cough and shortness of breath. Cases of severe infection can progress to severe pneumonia, multi-organ failure, and death. The time from exposure to onset of symptoms is approximately 2 to 14 days.

Standard methods for detecting viral infection may be used to detect SARS-CoV-2 infection, including but not limited to, assessment of patient symptoms and background and genetic tests such as reverse transcription-polymerase chain reaction (rRT-PCR). The test can be done on patient samples such as respiratory or blood samples.

SARS-CoV-2 Spike (S) protein: A class I fusion glycoprotein initially synthesized as a precursor protein of approximately 1270 amino acids in size. Individual precursor S polypeptides form a homotrimer and undergo glycosylation within the Golgi apparatus as well as processing to remove the signal peptide. The S polypeptide includes 51 and S2 proteins separated by a protease cleavage site between approximately position 685/68. Cleavage at this site generates separate 51 and S2 polypeptide chains, which remain associated as S1/S2 protomers within the homotrimer. The S1 subunit is distal to the virus membrane and contains the receptor-binding domain (RBD) that mediates virus attachment to its host receptor. The S2 subunit contains the fusion protein machinery, such as the fusion peptide, two heptad-repeat sequences (HR1 and HR2) and a central helix typical of fusion glycoproteins, a transmembrane domain, and the cytosolic tail domain.

The numbering used in the disclosed SARS-CoV-2 S proteins and fragments thereof is relative to the S protein of SARS-CoV-2, the sequence of which is provided as SEQ ID NO: 1, and deposited as NCBI Ref. No. YP_009724390.1, which is incorporated by reference herein in its entirety.

SARS-CoV-2 Spike (S) protein prefusion conformation: A structural conformation adopted by the ectodomain of the SARS-CoV-2 S protein following processing into a mature SARS-CoV-2 S protein in the secretory system, and prior to triggering of the fusogenic event that leads to transition of SARS-CoV-2 S to the postfusion conformation. The three-dimensional structure of an exemplary SARS-CoV-2 S protein in a prefusion conformation is disclosed herein (see Example 1).

A SARS-CoV-2 S ectodomain trimer “stabilized in a prefusion conformation” comprises one or more amino acid substitutions, deletions, or insertions compared to a native SARS-CoV-2 S sequence that provide for increased retention of the prefusion conformation compared to SARS-CoV-2 S ectodomain trimers formed from a corresponding native SARS-CoV-2 S sequence. The “stabilization” of the prefusion conformation by the one or more amino acid substitutions, deletions, or insertions can be, for example, energetic stabilization (for example, reducing the energy of the prefusion conformation relative to the post-fusion open conformation) and/or kinetic stabilization (for example, reducing the rate of transition from the prefusion conformation to the postfusion conformation). Additionally, stabilization of the SARS-CoV-2 S ectodomain trimer in the prefusion conformation can include an increase in resistance to denaturation compared to a corresponding native SARS-CoV-2 S sequence. Methods of determining if a SARS-CoV-2 S ectodomain trimer is in the prefusion conformation include (but are not limited to) negative-stain electron microscopy.

Sequence identity: The similarity between amino acid sequences is expressed in terms of the similarity between the sequences, otherwise referred to as sequence identity. Sequence identity is frequently measured in terms of percentage identity; the higher the percentage, the more similar the two sequences are. Homologs, orthologs, or variants of a polypeptide will possess a relatively high degree of sequence identity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Homologs and variants of a polypeptide (such as a SARS-CoV-2 S ectodomain) are typically characterized by possession of at least about 75%, for example at least about 80%, 85%, 90%, 91%, 92%, 93%, 94%, 95%, 96%, 97%, 98% or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet.

As used herein, reference to “at least 90% identity” or similar language refers to “at least 90%, at least 91%, at least 92%, at least 93%, at least 94%, at least 95%, at least 96%, at least 97%, at least 98%, at least 99%, or even 100% identity” to a specified reference sequence.

Signal Peptide: A short amino acid sequence (e.g., approximately 10-35 amino acids in length) that directs newly synthesized secretory or membrane proteins to and through membranes (for example, the endoplasmic reticulum membrane). Signal peptides are typically located at the N-terminus of a polypeptide and are removed by signal peptidases. Signal peptide sequences typically contain three common structural features: an N-terminal polar basic region (n-region), a hydrophobic core, and a hydrophilic c-region).

Single chain SARS-CoV-2 S ectodomain: A recombinant SARS-CoV-2 S ectodomain including the SARS-CoV-2 S1 and S2 proteins in a single contiguous polypeptide chain. Single chain SARS-CoV-2 S ectodomain can trimerize to form a SARS-CoV-2 S ectodomain trimer. A single SARS-CoV-2 S ectodomain includes mutations to prevent protease cleavage at the S₁/S₂ cleavage site. Therefore, when produced in cells, the SARS-CoV-2 S polypeptide is not cleaved into separate S₁ and S₂ polypeptide chains.

Soluble protein: A protein capable of dissolving in aqueous liquid at room temperature and remaining dissolved. The solubility of a protein may change depending on the concentration of the protein in the water-based liquid, the buffering condition of the liquid, the concentration of other solutes in the liquid, for example salt and protein concentrations, and the heat of the liquid. In several embodiments, a soluble protein is one that dissolves to a concentration of at least 0.5 mg/ml in phosphate buffered saline (pH 7.4) at room temperature and remains dissolved for at least 48 hours.

Subject: Living multi-cellular vertebrate organisms, a category that includes human and non-human mammals, such as non-human primates, pigs, camels, bats, sheep, cows, dogs, cats, rodents, and the like. In an example, a subject is a human. In an additional example, a subject is selected that is in need of inhibiting a SARS-CoV-2 infection. For example, the subject is either uninfected and at risk of the SARS-CoV-2 infection or is infected and in need of treatment.

T4 Fibritin trimerization domain: Also referred to as a “foldon” domain, the T4 Fibritin trimerization domain comprises an amino acid sequence that naturally forms a trimeric structure. In some examples, a T4 Fibritin trimerization domain can be linked to the C-terminus of a disclosed recombinant SARS-CoV-2 S protein ectodomain. In one example, a T4 Fibritin trimerization domain comprises the amino acid sequence set forth as (GYIPEAPRDGQAYVRKDGEWVLLSTF (SEQ ID NO: 6). In some embodiments, a protease cleavage site (such as a thrombin cleavage site) can be included between the C-terminus of the recombinant SARS-CoV-2 S ectodomain and the T4 Fibritin trimerization domain to facilitate removal of the trimerization domain as needed, for example, following expression and purification of the recombinant SARS-CoV-2 S ectodomain.

Transmembrane domain: An amino acid sequence that inserts into a lipid bilayer, such as the lipid bilayer of a cell or virus or virus-like particle. A transmembrane domain can be used to anchor an antigen to a membrane. In some examples a transmembrane domain is a SARS-CoV-2 S transmembrane domain.

Vaccine: A pharmaceutical composition that induces a prophylactic or therapeutic immune response in a subject. In some cases, the immune response is a protective immune response. Typically, a vaccine induces an antigen-specific immune response to an antigen of a pathogen, for example a viral pathogen, or to a cellular constituent correlated with a pathological condition. A vaccine may include a polynucleotide (such as a nucleic acid encoding a disclosed antigen), a peptide or polypeptide (such as a disclosed antigen), a virus, a cell or one or more cellular constituents. In a non-limiting example, a vaccine induces an immune response that reduces the severity of the symptoms associated with a SARS-CoV-2 infection and/or decreases the viral load compared to a control. In another non-limiting example, a vaccine induces an immune response that reduces and/or prevents a SARS-CoV-2 infection compared to a control.

Vector: An entity containing a DNA or RNA molecule bearing a promoter(s) that is operationally linked to the coding sequence of an antigen(s) of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant nucleic acid vectors having at least some nucleic acid sequences derived from one or more viruses.

Virus-like particle (VLP): A non-replicating, viral shell, derived from any of several viruses. VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. The presence of VLPs following recombinant expression of viral proteins can be detected using conventional techniques, such as by electron microscopy, biophysical characterization, and the like. Further, VLPs can be isolated by known techniques, e.g., density gradient centrifugation and identified by characteristic density banding. See, for example, Baker et al. (1991) Biophys. J. 60:1445-1456; and Hagensee et al. (1994) J. Virol. 68:4503-4505; Vincente, J Invertebr Pathol., 2011; Schneider-Ohrum and Ross, Curr. Top. Microbiol. Immunol., 354: 53073, 2012).

### II. RECOMBINANT SARS-COV-2 SPIKE PROTEINS

Disclosed herein are recombinant SARS-CoV-2 S ectodomain trimers comprising protomers comprising one or more amino acid substitutions that inhibit a conformational change in the SARS-CoV-2 S protein from the prefusion conformation to the postfusion conformation, and therefore stabilize the SARS-CoV-2 S ectodomain trimer in the prefusion conformation. The recombinant SARS-CoV-2 S ectodomain trimer produces a superior immune response compared to corresponding SARS-CoV-2 S ectodomain trimer that is not stabilized in the prefusion conformation.

An exemplary sequence of native SARS-CoV-2 S protein (including the ectodomain and TM and CT domains) is provided as SEQ ID NO: 1 (NCBI Ref. No. YP_009724390.1, incorporated by reference herein):

MEVFLVLLPLVSSQCVNLTTRTQLPPAYTNSFTRGVYYPDKVFRSSVLHS
TQDLELPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

The amino acid numbering used herein for residues of the SARS-CoV-2 S protein is with reference to the SARS-CoV-2 S sequence provided as SEQ ID NO: 1. With reference to the SARS-CoV-2 S protein sequence provided as SEQ ID NO: 1, the ectodomain of the SARS-CoV-2 S protein includes about residues 16-1208. Residues 1-15 are the signal peptide, which is removed during cellular processing. The S1/S2 cleavage site is located at position 685/686. The HR1 is located at about residues 915-983. The central helix is located at about residues 988-1029. The HR2 is located at about 1162-1194. The C-terminal end of the S2 ectodomain is located at about residue 1208. In some embodiments, the protomers of the prefusion-stabilized SARS-CoV-2 S ectodomain trimer can have a C-terminal residue (which can be linked to a trimerization domain, or a transmembrane domain, for example) of the C-terminal residue of the HR2 (e.g., position 1194), or the ectodomain (e.g., position 1208), or from one of positions 1194-1208. The position numbering of the S protein may vary between SARS-CoV-2 strains, but the sequences can be aligned to determine relevant structural domains and cleavage sites. It will be appreciated that a few residues (such as up to 10) on the N- and C-terminal ends of the ectodomain can be removed or modified in the disclosed immunogens without decreasing the utility of the S ectodomain trimer as an immunogen.

In some embodiments, the immunogen comprises a recombinant SARS-CoV-2 S ectodomain trimer comprising protomers comprising one or more (such as two, for example two consecutive) amino acid substitutions at or near the boundary between a HR1 domain and a central helix domain that stabilize the S ectodomain trimer in the prefusion conformation, wherein the amino acid substitutions are glycine and/or proline substitutions. In some such embodiments, the one or more (such as two, for example two consecutive) amino acid substitutions that stabilize the S ectodomain in the prefusion conformation are located between a position 15 amino acids N-terminal of a C-terminal residue of the HR1 and a position 5 amino acids C-terminal of a N-terminal residue of the central helix. In some embodiments, the one or more (such as two, for example two consecutive) amino acid substitutions that stabilize the SARS-CoV-2 S ectodomain trimer in the prefusion conformation are located between residues 975 to 995 (such as 981-992) of the S ectodomain protomers in the trimer, wherein the amino acid substitutions are glycine and/or proline substitutions. In some embodiments, the SARS-CoV-2 S ectodomain trimer is stabilized in the prefusion conformation by glycine and/or proline substitutions at positions D985, K986, and/or V987 of the S ectodomain protomers in the trimer.

In some embodiments, the immunogen comprises a recombinant SARS-CoV-2 S ectodomain trimer comprising protomers comprising one or more (such as two, for example two consecutive) proline substitutions at or near the boundary between a HR1 domain and a central helix domain that stabilize the S ectodomain trimer in the prefusion conformation. In some such embodiments, the one or more (such as two, for example two consecutive) proline substitutions that stabilize the S ectodomain in the prefusion conformation are located between a position 15 amino acids N-terminal of a C-terminal residue of the HR1 and a position 5 amino acids C-terminal of a N-terminal residue of the central helix.

In some embodiments, the one or more (such as two, for example two consecutive) proline substitutions that stabilize the SARS-CoV-2 S ectodomain trimer in the prefusion conformation are located between residues 975 to 995 (such as 981-992) of the S ectodomain protomers in the trimer. In some embodiments, the SARS-CoV-2 S ectodomain trimer is stabilized in the prefusion conformation by K986P and V987P substitutions (“2P”) in the S ectodomain protomers in the trimer. In some embodiments, the SARS-CoV-2 S ectodomain trimer is stabilized in the prefusion conformation by one or two proline substitutions at positions D985, K986, or V987 of the S ectodomain protomers in the trimer.

In some embodiments, the protomers of the recombinant SARS-CoV-2 S ectodomain trimer stabilized in the prefusion conformation by the one or more proline substitutions (such as K986P and V987P substitutions) comprise one or more additional modifications for stabilization in the prefusion conformation.

In some embodiments, the C-terminal residue of the ectodomains of the protomers in the recombinant SARS-CoV-2 S ectodomain trimer can be linked to a trimerization domain to promote trimerization of the protomers, and to stabilize the membrane proximal aspect of the protomers in a trimeric configuration. Non-limiting examples of exogenous multimerization domains that promote stable trimers of soluble recombinant proteins include: the GCN4 leucine zipper (Harbury et al. 1993 Science 262:1401-1407), the trimerization motif from the lung surfactant protein (Hoppe et al. 1994 FEBS Lett 344:191-195), collagen (McAlinden et al. 2003 J Biol Chem 278:42200-42207), and the phage T4 fibritin (Miroshnikov et al. 1998 Protein Eng 11:329-414), any of which can be linked to a recombinant SARS-CoV-2 S ectodomain described herein (e.g., by linkage to the C-terminus of S2 ectodomain) to promote trimerization of the recombinant SARS-CoV-2 S ectodomain.

In some examples, the C-terminal residue of the S2 ectodomain can be linked to a T4 fibritin domain. In specific examples, the T4 fibritin domain can include the amino acid sequence GYIPEAPRDGQAYVRKDGEWVLLSTF (SEQ ID NO: 6), which adopts a β-propeller conformation, and can fold and trimerize in an autonomous way (Tao et al. 1997 Structure 5:789-798).

Optionally, the heterologous trimerization is connected to the recombinant SARS-CoV-2 S ectodomain via a peptide linker, such as an amino acid linker. Non-limiting examples of peptide linkers that can be used include glycine, serine, and glycine-serine linkers.

An exemplary sequence of SARS-CoV-2 S ectodomain including a double proline substitution for stabilization in the prefusion conformation and linked to a T4 fibritin trimerization domain is provided as SEQ ID NO: 2:

MEVFLVLLPLVSSQCVNLTTRTQLPPAYTNSFTRGVYYPDKVFRSSVLHS
TQDLELPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGE
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQGGYIPEAPRDGQAYVRKDGEWVLLSTF

In some embodiments, the recombinant SARS-CoV-2 S ectodomain trimer comprises protomers comprising the ectodomain sequence of SEQ ID NO: 2. In some embodiments, the recombinant SARS-CoV-2 S ectodomain trimer comprises protomers comprising residues 16-1208 of SEQ ID NO: 2. In some embodiments, the recombinant SARS-CoV-2 S ectodomain trimer comprises protomers comprising a sequence at least 90% (such as at least 95%, at least 98%, or at least 99%) identical to the ectodomain sequence of SEQ ID NO: 2, wherein the SARS-CoV-2 S ectodomain trimer is stabilized in the prefusion conformation with one or more of the modifications provided herein (such as the K986P and V987P substitutions). In some embodiments, the recombinant SARS-CoV-2 S ectodomain trimer comprises protomers comprising a sequence at least 90% (such as at least 95%, at least 98%, or at least 99%) identical residues 16-1208 SEQ ID NO: 2, wherein the SARS-CoV-2 S ectodomain trimer is stabilized in the prefusion conformation with one or more of the modifications provided herein (such as the K986P and V987P substitutions).

In some embodiments, the recombinant SARS-CoV-2 S ectodomain trimer comprises protomers comprising the ectodomain sequence of SEQ ID NO: 2 that are each linked to a trimerization domain, such as a T4 Fibritin trimerization domain. In some embodiments, the recombinant SARS-CoV-2 S ectodomain trimer comprises protomers linked to a trimerization domain comprising residues 16-1235 of SEQ ID NO: 2. In some embodiments, the recombinant SARS-CoV-2 S ectodomain trimer comprises protomers linked to a trimerization domain comprising a sequence at least 90% (such as at least 95%, at least 98%, or at least 99%) identical to residues 16-1235 of SEQ ID NO: 2, wherein the SARS-CoV-2 S ectodomain trimer is stabilized in the prefusion conformation with one or more of the modifications provided herein (such as the K986P and V987P substitutions).

In some embodiments, the SARS-CoV-2 S ectodomain trimer can be membrane anchored, for example, for embodiments where the SARS-CoV-2 S ectodomain trimer is expressed as an attenuated viral vaccine, or a virus like particle, or by recombinant nucleic acid (such as mRNA). In such embodiments, the protomers in the trimer typically each comprise a C-terminal linkage to a transmembrane domain, such as the transmembrane domain (and optionally the cytosolic tail) of SARS-CoV-2 S protein. In some embodiments, one or more peptide linkers (such as a gly-ser linker, for example, a 10 amino acid glycine-serine peptide linker can be used to link the recombinant SARS-CoV-2 S ectodomain protomer to the transmembrane domain. The protomers linked to the transmembrane domain can include any of the stabilizing mutations provided herein (or combinations thereof) as long as the recombinant SARS-CoV-2 S ectodomain trimer linked to the transmembrane domain retains the desired properties (e.g., the SARS-CoV-2 S prefusion conformation).

An exemplary sequence of SARS-CoV-2 S protein (including the ectodomain and TM and CT domains) including a double proline substitution for stabilization in the prefusion conformation is provided as SEQ ID NO: 3:

MEVFLVLLPLVSSQCVNLTTRTQLPPAYTNSFTRGVYYPDKVFRSSVLHS
TQDLELPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

In some embodiments, the recombinant SARS-CoV-2 S ectodomain trimer comprises protomers comprising the ectodomain sequence of SEQ ID NO: 3 that are each linked to a transmembrane domain and or a cytoplasmic tail. In some embodiments, the recombinant SARS-CoV-2 S ectodomain trimer comprises protomers linked to a transmembrane domain comprising residues 16-1273 of SEQ ID NO: 3. In some embodiments, the recombinant SARS-CoV-2 S ectodomain trimer comprises protomers linked to a transmembrane domain comprising a sequence at least 90% (such as at least 95%, at least 98%, or at least 99%) identical to residues 16-1273 of SEQ ID NO: 3, wherein the SARS-CoV-2 S ectodomain trimer is stabilized in the prefusion conformation with one or more of the modifications provided herein (such as the K986P and V987P substitutions).

In some embodiments, the SARS-CoV-2 S ectodomain trimer can be composed of three single-chain SARS-CoV-2 S ectodomain protomers, each including a single polypeptide chain including the 51 protein and S2 ectodomain. Single chain SARS-CoV-2 S ectodomain protomers can be generated by mutating the S1/S2 protease cleavage site to prevent cleavage and formation of distinct 51 and S2 polypeptide chains. In some embodiments, the 51 and S2 polypeptides in the single chain SARS-CoV-2 S ectodomain protomers are joined by a linker, such as a peptide linker. Examples of peptide linkers that can be used include glycine, serine, and glycine-serine linkers. Any of the stabilizing mutations (or combinations thereof) disclosed herein can be included in the single chain SARS-CoV-2 S ectodomain protomers as long as the SARS-CoV-2 S ectodomain trimer composed of such protomers retains the desired properties (e.g., the prefusion conformation).

An exemplary sequence of single chain SARS-CoV-2 S ectodomain including a double proline substitution for stabilization in the prefusion conformation and linked to a T4 fibritin trimerization domain is provided as SEQ ID NO: 4:

MEVFLVLLPLVSSQCVNLTTRTQLPPAYTNSFTRGVYYPDKVFRSSVLHS
TQDLELPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPGGSVASQSIIAYTMSLGAE
NSVAYSNNSIAIPTNFTISVTTEILPVSMTKTSVDCTMYICGDSTECSNL
LLQYGSFCTQLNRALTGIAVEQDKNTQEVFAQVKQIYKTPPIKDFGGFNF
SQILPDPSKPSKRSFIEDLLFNKVTLADAGFIKQYGDCLGDIAARDLICA
QKFNGLTVLPPLLTDEMIAQYTSALLAGTITSGWTFGAGAALQIPFAMQM
AYRFNGIGVTQNVLYENQKLIANQFNSAIGKIQDSLSSTASALGKLQDVV
NQNAQALNTLVKQLSSNFGAISSVLNDILSRLDPPEAEVQIDRLITGRLQ
SLQTYVTQQLIRAAEIRASANLAATKMSECVLGQSKRVDFCGKGYHLMSF
PQSAPHGVVFLHVTYVPAQEKNFTTAPAICHDGKAHFPREGVFVSNGTHW
FVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQE
LGKYEQGGYIPEAPRDGQAYVRKDGEWVLLSTF

In some embodiments, the recombinant single chain SARS-CoV-2 S ectodomain trimer comprises protomers comprising the ectodomain sequence of SEQ ID NO: 4 linked to a trimerization domain such as a T4 Fibritin trimerization domain. In some embodiments, the recombinant single chain SARS-CoV-2 S ectodomain trimer linked to the transmembrane domain comprises protomers comprising residues 16-1233 of SEQ ID NO: 4. In some embodiments, the recombinant single chain SARS-CoV-2 S ectodomain trimer linked to the transmembrane domain comprises protomers comprising a sequence at least 90% (such as at least 95%, at least 98%, or at least 99%) identical to residues 16-1233 of SEQ ID NO: 4, wherein the SARS-CoV-2 S ectodomain trimer is stabilized in the prefusion conformation with one or more of the modifications provided herein (such as the K986P and V987P substitutions).

An exemplary sequence of single chain SARS-CoV-2 S protein (including the ectodomain and TM and CT domains) including a double proline substitution for stabilization in the prefusion conformation is provided as SEQ ID NO: 5:

MEVFLVLLPLVSSQCVNLTTRTQLPPAYTNSFTRGVYYPDKVFRSSVLHS
TQDLELPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPGGSVASQSIIAYTMSLGAE
NSVAYSNNSIAIPTNFTISVTTEILPVSMTKTSVDCTMYICGDSTECSNL
LLQYGSFCTQLNRALTGIAVEQDKNTQEVFAQVKQIYKTPPIKDFGGFNF
SQILPDPSKPSKRSFIEDLLFNKVTLADAGFIKQYGDCLGDIAARDLICA
QKFNGLTVLPPLLTDEMIAQYTSALLAGTITSGWTFGAGAALQIPFAMQM
AYRFNGIGVTQNVLYENQKLIANQFNSAIGKIQDSLSSTASALGKLQDVV
NQNAQALNTLVKQLSSNFGAISSVLNDILSRLDPPEAEVQIDRLITGRLQ
SLQTYVTQQLIRAAEIRASANLAATKMSECVLGQSKRVDFCGKGYHLMSF
PQSAPHGVVFLHVTYVPAQEKNFTTAPAICHDGKAHFPREGVFVSNGTHW
FVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQE
LGKYEQYIKWPWYIWLGFIAGLIAIVMVTIMLCCMTSCCSCLKGCCSCGS
CCKFDEDDSEPVLKGVKLHYT

In some embodiments, the recombinant single chain SARS-CoV-2 S ectodomain trimer comprises protomers comprising the ectodomain sequence of SEQ ID NO: 5 that are each linked to a transmembrane domain and or a cytoplasmic tail. In some embodiments, the recombinant single chain SARS-CoV-2 S ectodomain trimer comprises protomers linked to a transmembrane domain comprising residues 16-1271 of SEQ ID NO: 5. In some embodiments, the recombinant single chain SARS-CoV-2 S ectodomain trimer comprises protomers linked to a transmembrane domain comprising a sequence at least 90% (such as at least 95%, at least 98%, or at least 99%) identical to residues 16-1271 of SEQ ID NO: 5, wherein the SARS-CoV-2 S ectodomain trimer is stabilized in the prefusion conformation with one or more of the modifications provided herein (such as the K986P and V987P substitutions).

In some embodiments, the protomers in the recombinant SARS-CoV-2 S ectodomain trimer comprise the K986P and V987P substitutions for prefusion stabilization and further comprise one or more of N501Y, K417N, and E484K substitutions. For example, the protomers in the recombinant SARS-CoV-2 S ectodomain trimer comprise the K986P and V987P substitutions and further comprise a N501Y substitution, a K417N substitution, a E484K substitution, N501Y and K417N substitutions, K417N and E484K substitutions, N501Y and E484K substitutions, or N501Y, K417N, and E484K substitutions.

The recombinant SARS-CoV-2 S ectodomain trimer and variants thereof can be produced using recombinant techniques, or chemically or enzymatically synthesized.

Analogs and variants of the recombinant SARS-CoV-2 S ectodomain trimer may be used in the methods and systems of the present disclosure. Through the use of recombinant DNA technology, variants of the recombinant SARS-CoV-2 S ectodomain trimer may be prepared by altering the underlying DNA. All such variations or alterations in the structure of the recombinant SARS-CoV-2 S ectodomain trimer resulting in variants are included within the scope of this disclosure. Such variants include insertions, substitutions, or deletions of one or more amino acid residues, glycosylation variants, unglycosylated recombinant SARS-CoV-2 S ectodomain trimer, organic and inorganic salts, covalently modified derivatives of the recombinant SARS-CoV-2 S ectodomain trimer, or a precursor thereof. Such variants may maintain one or more of the functional, biological activities of the recombinant SARS-CoV-2 S ectodomain trimer, such as binding to cell surface receptor. The recombinant SARS-CoV-2 S ectodomain trimer thereof can be modified, for example, by PEGylation, to increase the half-life of the protein in the recipient, and/or to make the protein more stable for delivery to a subject.

In some embodiments, a recombinant SARS-CoV-2 S ectodomain trimer useful within the disclosure is modified by replacement of one or more naturally occurring side chains of the 20 genetically encoded amino acids (or D-amino acids) with other side chains, for example with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclics. For example, proline analogs can be made in which the ring size of the proline residue is changed from a 5-membered ring to a 4-, 6-, or 7-membered ring. Cyclic groups can be saturated or unsaturated, and if unsaturated, can be aromatic or non-aromatic. Heterocyclic groups can contain one or more nitrogen, oxygen, and/or sulphur heteroatoms.

### Protein Nanoparticles

In some embodiments a protein nanoparticle (such as a self-assembling protein nanoparticle) is provided that includes a recombinant SARS-CoV-2 S ectodomain trimer displayed on its surface. Non-limiting example of self-assembling protein nanoparticles include ferritin nanoparticles, encapsulin nanoparticles, Sulfur Oxygenase Reductase (SOR) nanoparticles, and lumazine synthase nanoparticles, which are comprised of an assembly of monomeric subunits including ferritin proteins, encapsulin proteins, SOR proteins, and lumazine synthase, respectively. Additional protein nanoparticle structures are described by Heinze et al., J Phys Chem B., 120(26):5945-52, 2016; Hsia et al., Nature, 535(7610):136-9, 2016; and King et al., Nature, 510(7503):103-8, 2014; each of which is incorporated by reference herein.

In several embodiments, to construct such protein nanoparticles, nucleic acid encoding a protomer of the SARS-CoV-2 S ectodomain trimer can be fused to nucleic acid encoding a subunit of the protein nanoparticle (such as a ferritin protein, an encapsulin protein, a SOR protein, or a lumazine synthase protein) and expressed in cells under appropriate conditions. The fusion protein self-assembles into a nanoparticle any can be purified.

In several embodiments, to construct such protein nanoparticles, a purified SARS-CoV-2 S ectodomain trimer can be linked (for example, via bioconjugation) to subunits of a purified self-assembling protein nanoparticle (such as a ferritin protein, an encapsulin protein, a SOR protein, or a lumazine synthase protein) and the resulting nanoparticle/S trimer purified.

In some embodiments, the SARS-CoV-2 S ectodomain trimer is included in a self-assembling protein nanocage that directs its own release from cells inside small vesicles in a manner that resembles viruses, for example, as described in Votteler et al., “Designed proteins induce the formation of nanocage-containing extracellular vesicles,” Nature 540, 292-29, 2016. This hybrid biomaterial can fuse its membranes with target cells and deliver its contents, thereby transferring cargoes from one cell to another.

In some embodiments, a protomer of a disclosed recombinant SARS-CoV-2 S ectodomain trimer can be linked to a ferritin subunit to construct a ferritin nanoparticle. Ferritin nanoparticles and their use for immunization purposes (e.g., for immunization against influenza antigens) have been disclosed in the art (see, e.g., Kanekiyo et al., Nature, 499:102-106, 2013, incorporated by reference herein in its entirety). Ferritin is a globular protein that is found in all animals, bacteria, and plants, and which acts primarily to control the rate and location of polynuclear Fe(III)₂O₃ formation through the transportation of hydrated iron ions and protons to and from a mineralized core. The globular form of the ferritin nanoparticle is made up of monomeric subunits, which are polypeptides having a molecule weight of approximately 17-20 kDa. An example of the amino acid sequence of one such monomeric ferritin subunit is represented by:

(SEQ ID NO: 7)
ESQVRQQFSKDIEKLLNEQVNKEMQSSNLYMSMSSWCYTHSLDGAGLFLF
DHAAEEYEHAKKLIIFLNENNVPVQLTSISAPEHKFEGLTQIFQKAYEHE
QHISESINNIVDHAIKSKDHATFNFLQWYVAEQHEEEVLFKDILDKIELI
GNENHGLYLADQYVKGIAKSRKS

Each monomeric subunit has the topology of a helix bundle which includes a four antiparallel helix motif, with a fifth shorter helix (the c-terminal helix) lying roughly perpendicular to the long axis of the 4 helix bundle. According to convention, the helices are labeled ‘A, B, C, D & E’ from the N-terminus respectively. The N-terminal sequence lies adjacent to the capsid three-fold axis and extends to the surface, while the E helices pack together at the four-fold axis with the C-terminus extending into the capsid core. The consequence of this packing creates two pores on the capsid surface. It is expected that one or both of these pores represent the point by which the hydrated iron diffuses into and out of the capsid. Following production, these monomeric subunit proteins self-assemble into the globular ferritin protein. Thus, the globular form of ferritin comprises 24 monomeric, subunit proteins, and has a capsid-like structure having 432 symmetry. Methods of constructing ferritin nanoparticles are known to the person of ordinary skill in the art and are further described herein (see, e.g., Zhang, Int. J. Mol. Sci., 12:5406-5421, 2011, which is incorporated herein by reference in its entirety).

In specific examples, the ferritin polypeptide is E. coli ferritin, Helicobacter pylori ferritin, human light chain ferritin, bullfrog ferritin or a hybrid thereof, such as E. coli-human hybrid ferritin, E. coli-bullfrog hybrid ferritin, or human-bullfrog hybrid ferritin. Exemplary amino acid sequences of ferritin polypeptides and nucleic acid sequences encoding ferritin polypeptides for use to make a ferritin nanoparticle including a recombinant SARS-CoV-2 S ectodomain can be found in GENBANK®, for example at accession numbers ZP_03085328, ZP_06990637, EJB64322.1, AAA35832, NP_000137 AAA49532, AAA49525, AAA49524 and AAA49523, which are specifically incorporated by reference herein in their entirety as available Apr. 10, 2015. In some embodiments, a recombinant SARS-CoV-2 S ectodomain can be linked to a ferritin subunit including an amino acid sequence at least 80% (such as at least 85%, at least 90%, at least 95%, or at least 97%) identical to amino acid sequence set forth as SEQ ID NO: 8.

In some embodiments, a protomer of a disclosed recombinant SARS-CoV-2 S ectodomain trimer can be linked to a lumazine synthase subunit to construct a lumazine synthase nanoparticle. The globular form of lumazine synthase nanoparticle is made up of monomeric subunits; an example of the sequence of one such lumazine synthase subunit is provides as the amino acid sequence set forth as:

(SEQ ID NO: 8)
MQIYEGKLTAEGLRFGIVASRFNHALVDRLVEGAIDAIVRHGGREEDITL
VRVPGSWEIPVAAGELARKEDIDAVIAIGVLIRGATPHFDYIASEVSKGL
ADLSLELRKPITFGVITADTLEQAIERAGTKHGNKGWEAALSAIEMANLF
KSLR.

In some embodiments, a protomer of a disclosed recombinant SARS-CoV-2 S ectodomain trimer can be linked to a lumazine synthase subunit including an amino acid sequence at least 80% (such as at least 85%, at least 90%, at least 95%, or at least 97%) identical to amino acid sequence set forth as SEQ ID NO: 8.

In some embodiments, a protomer of a disclosed recombinant SARS-CoV-2 S ectodomain trimer can be linked to an encapsulin nanoparticle subunit to construct an encapsulin nanoparticle. The globular form of the encapsulin nanoparticle is made up of monomeric subunits; an example of the sequence of one such encapsulin subunit is provides as the amino acid sequence set forth as

(SEQ ID NO: 9)
MEFLKRSFAPLTEKQWQEIDNRAREIFKTQLYGRKFVDVEGPYGWEYAAH
PLGEVEVLSDENEVVKWGLRKSLPLIELRATFTLDLWELDNLERGKPNVD
LSSLEETVRKVAEFEDEVIFRGCEKSGVKGLLSFEERKIECGSTPKDLLE
AIVRALSIFSKDGIEGPYTLVINTDRWINFLKEEAGHYPLEKRVEECLRG
GKIITTPRIEDALVVSERGGDFKLILGQDLSIGYEDREKDAVRLFITETF
TFQVVNPEALILLKF.

In some embodiments, a protomer of a disclosed recombinant SARS-CoV-2 S ectodomain trimer can be linked to an encapsulin subunit including an amino acid sequence at least 80% (such as at least 85%, at least 90%, at least 95%, or at least 97%) identical to amino acid sequence set forth as SEQ ID NO: 9. Encapsulin proteins are a conserved family of bacterial proteins also known as linocin-like proteins that form large protein assemblies that function as a minimal compartment to package enzymes. The encapsulin assembly is made up of monomeric subunits, which are polypeptides having a molecule weight of approximately 30 kDa. Following production, the monomeric subunits self-assemble into the globular encapsulin assembly including 60, or in some cases, 180 monomeric subunits. Methods of constructing encapsulin nanoparticles are known to the person of ordinary skill in the art, and further described herein (see, for example, Sutter et al., Nature Struct. and Mol. Biol., 15:939-947, 2008, which is incorporated by reference herein in its entirety). In specific examples, the encapsulin polypeptide is bacterial encapsulin, such as Thermotoga maritime or Pyrococcus furiosus or Rhodococcus erythropolis or Myxococcus xanthus encapsulin.

In some embodiments, a protomer of a disclosed recombinant SARS-CoV-2 S ectodomain trimer can be linked to a Sulfur Oxygenase Reductase (SOR) subunit to construct a recombinant SOR nanoparticle. In some embodiments, the SOR subunit can include the amino acid sequence set forth as

(SEQ ID NO: 10)
MEFLKRSFAPLTEKQWQEIDNRAREIFKTQLYGRKFVDVEGPYGWEYAAH
PLGEVEVLSDENEVVKWGLRKSLPLIELRATFTLDLWELDNLERGKPNVD
LSSLEETVRKVAEFEDEVIFRGCEKSGVKGLLSFEERKIECGSTPKDLLE
AIVRALSIFSKDGIEGPYTLVINTDRWINFLKEEAGHYPLEKRVEECLRG
GKIITTPRIEDALVVSERGGDFKLILGQDLSIGYEDREKDAVRLFITETF
TFQVVNPEALILLKF.

In some embodiments, a protomer of a disclosed recombinant SARS-CoV-2 S ectodomain trimer can be linked to a SOR subunit including an amino acid sequence at least 80% (such as at least 85%, at least 90%, at least 95%, or at least 97%) identical to amino acid sequence set forth as SEQ ID NO: 10.

SOR proteins are microbial proteins (for example from the thermoacidophilic archaeon Acidianus ambivalens that form 24 subunit protein assemblies. Methods of constructing SOR nanoparticles are known to the person of ordinary skill in the art (see, e.g., Urich et al., Science, 311:996-1000, 2006, which is incorporated by reference herein in its entirety). An example of an amino acid sequence of a SOR protein for use to make SOR nanoparticles is set forth in Urich et al., Science, 311:996-1000, 2006, which is incorporated by reference herein in its entirety.

For production purposes, in some embodiments, the recombinant SARS-CoV-2 S ectodomain linked to the nanoparticle subunit can include an N-terminal signal peptide that is cleaved during cellular processing. For example, the recombinant SARS-CoV-2 S ectodomain protomer linked to the protein nanoparticle subunit can include a signal peptide at its N-terminus including, for example, a native coronavirus S signal peptide

The protein nanoparticles can be expressed in appropriate cells (e.g., HEK 293 Freestyle cells) and fusion proteins are secreted from the cells self-assembled into nanoparticles. The nanoparticles can be purified using known techniques, for example by a few different chromatography procedures, e.g. Mono Q (anion exchange) followed by size exclusion (SUPEROSE® 6) chromatography.

Several embodiments include a monomeric subunit of a ferritin, encapsulin, SOR, or lumazine synthase protein, or any portion thereof which is capable of directing self-assembly of monomeric subunits into the globular form of the protein Amino acid sequences from monomeric subunits of any known ferritin, encapsulin, SOR, or lumazine synthase protein can be used to produce fusion proteins with the recombinant SARS-CoV-2 S ectodomain or immunogenic fragment thereof, so long as the monomeric subunit is capable of self-assembling into a nanoparticle displaying the recombinant SARS-CoV-2 S ectodomain or immunogenic fragment thereof on its surface.

The fusion proteins need not comprise the full-length sequence of a monomeric subunit polypeptide of a ferritin, encapsulin, SOR, or lumazine synthase protein. Portions, or regions, of the monomeric subunit polypeptide can be utilized so long as the portion comprises amino acid sequences that direct self-assembly of monomeric subunits into the globular form of the protein.

### III. POLYNUCLEOTIDES AND EXPRESSION

Polynucleotides encoding a protomer of any of the disclosed recombinant S ectodomain trimers are also provided. These polynucleotides include DNA, cDNA and RNA sequences which encode the protomer, as well as vectors including the DNA, cDNA and RNA sequences, such as a DNA or RNA vector used for immunization. The genetic code to construct a variety of functionally equivalent nucleic acids, such as nucleic acids which differ in sequence but which encode the same protein sequence, or encode a conjugate or fusion protein including the nucleic acid sequence.

An exemplary nucleic acid sequence encoding SARS-CoV-2 S protein is provided as SEQ ID NO: 11:

ATGTTTGTTTTTCTTGTTTTATTGCCACTAGTGTGTAGTCAGTGTGTTAA
TCTTACAACCAGAACTCAATTACCCCCTGCATACACTAATTCTTTCACAC
GTGGTGTTTATTACCCTGACAAAGTTTTCAGATCCTCAGTTTTACATTCA
ACTCAGGACTTGTTCTTACCTTTCTTTTCCAATGTTACTTGGTTCCATGC
TATACATGTCTCTGGGACCAATGGTACTAAGAGGTTTGATAACCCTGTCC
TACCATTTAATGATGGTGTTTATTTTGCTTCCACTGAGAAGTCTAACATA
ATAAGAGGCTGGATTTTTGGTACTACTTTAGATTCGAAGACCCAGTCCCT
ACTTATTGTTAATAACGCTACTAATGTTGTTATTAAAGTCTGTGAATTTC
AATTTTGTAATGATCCATTTTTGGGTGTTTATTACCACAAAAACAACAAA
AGTTGGATGGAAAGTGAGTTCAGAGTTTATTCTAGTGCGAATAATTGCAC
TTTTGAATATGTCTCTCAGCCTTTTCTTATGGACCTTGAAGGAAAACAGG
GTAATTTCAAAAATCTTAGGGAATTTGTGTTTAAGAATATTGATGGTTAT
TTTAAAATATATTCTAAGCACACGCCTATTAATTTAGTGCGTGATCTCCC
TCAGGGTTTTTCGGCTTTAGAACCATTGGTAGATTTGCCAATAGGTATTA
ACATCACTAGGTTTCAAACTTTACTTGCTTTACATAGAAGTTATTTGACT
CCTGGTGATTCTTCTTCAGGTTGGACAGCTGGTGCTGCAGCTTATTATGT
GGGTTATCTTCAACCTAGGACTTTTCTATTAAAATATAATGAAAATGGAA
CCATTACAGATGCTGTAGACTGTGCACTTGACCCTCTCTCAGAAACAAAG
TGTACGTTGAAATCCTTCACTGTAGAAAAAGGAATCTATCAAACTTCTAA
CTTTAGAGTCCAACCAACAGAATCTATTGTTAGATTTCCTAATATTACAA
ACTTGTGCCCTTTTGGTGAAGTTTTTAACGCCACCAGATTTGCATCTGTT
TATGCTTGGAACAGGAAGAGAATCAGCAACTGTGTTGCTGATTATTCTGT
CCTATATAATTCCGCATCATTTTCCACTTTTAAGTGTTATGGAGTGTCTC
CTACTAAATTAAATGATCTCTGCTTTACTAATGTCTATGCAGATTCATTT
GTAATTAGAGGTGATGAAGTCAGACAAATCGCTCCAGGGCAAACTGGAAA
GATTGCTGATTATAATTATAAATTACCAGATGATTTTACAGGCTGCGTTA
TAGCTTGGAATTCTAACAATCTTGATTCTAAGGTTGGTGGTAATTATAAT
TACCTGTATAGATTGTTTAGGAAGTCTAATCTCAAACCTTTTGAGAGAGA
TATTTCAACTGAAATCTATCAGGCCGGTAGCACACCTTGTAATGGTGTTG
AAGGTTTTAATTGTTACTTTCCTTTACAATCATATGGTTTCCAACCCACT
AATGGTGTTGGTTACCAACCATACAGAGTAGTAGTACTTTCTTTTGAACT
TCTACATGCACCAGCAACTGTTTGTGGACCTAAAAAGTCTACTAATTTGG
TTAAAAACAAATGTGTCAATTTCAACTTCAATGGTTTAACAGGCACAGGT
GTTCTTACTGAGTCTAACAAAAAGTTTCTGCCTTTCCAACAATTTGGCAG
AGACATTGCTGACACTACTGATGCTGTCCGTGATCCACAGACACTTGAGA
TTCTTGACATTACACCATGTTCTTTTGGTGGTGTCAGTGTTATAACACCA
GGAACAAATACTTCTAACCAGGTTGCTGTTCTTTATCAGGATGTTAACTG
CACAGAAGTCCCTGTTGCTATTCATGCAGATCAACTTACTCCTACTTGGC
GTGTTTATTCTACAGGTTCTAATGTTTTTCAAACACGTGCAGGCTGTTTA
ATAGGGGCTGAACATGTCAACAACTCATATGAGTGTGACATACCCATTGG
TGCAGGTATATGCGCTAGTTATCAGACTCAGACTAATTCTCCTCGGCGGG
CACGTAGTGTAGCTAGTCAATCCATCATTGCCTACACTATGTCACTTGGT
GCAGAAAATTCAGTTGCTTACTCTAATAACTCTATTGCCATACCCACAAA
TTTTACTATTAGTGTTACCACAGAAATTCTACCAGTGTCTATGACCAAGA
CATCAGTAGATTGTACAATGTACATTTGTGGTGATTCAACTGAATGCAGC
AATCTTTTGTTGCAATATGGCAGTTTTTGTACACAATTAAACCGTGCTTT
AACTGGAATAGCTGTTGAACAAGACAAAAACACCCAAGAAGTTTTTGCAC
AAGTCAAACAAATTTACAAAACACCACCAATTAAAGATTTTGGTGGTTTT
AATTTTTCACAAATATTACCAGATCCATCAAAACCAAGCAAGAGGTCATT
TATTGAAGATCTACTTTTCAACAAAGTGACACTTGCAGATGCTGGCTTCA
TCAAACAATATGGTGATTGCCTTGGTGATATTGCTGCTAGAGACCTCATT
TGTGCACAAAAGTTTAACGGCCTTACTGTTTTGCCACCTTTGCTCACAGA
TGAAATGATTGCTCAATACACTTCTGCACTGTTAGCGGGTACAATCACTT
CTGGTTGGACCTTTGGTGCAGGTGCTGCATTACAAATACCATTTGCTATG
CAAATGGCTTATAGGTTTAATGGTATTGGAGTTACACAGAATGTTCTCTA
TGAGAACCAAAAATTGATTGCCAACCAATTTAATAGTGCTATTGGCAAAA
TTCAAGACTCACTTTCTTCCACAGCAAGTGCACTTGGAAAACTTCAAGAT
GTGGTCAACCAAAATGCACAAGCTTTAAACACGCTTGTTAAACAACTTAG
CTCCAATTTTGGTGCAATTTCAAGTGTTTTAAATGATATCCTTTCACGTC
TTGACAAAGTTGAGGCTGAAGTGCAAATTGATAGGTTGATCACAGGCAGA
CTTCAAAGTTTGCAGACATATGTGACTCAACAATTAATTAGAGCTGCAGA
AATCAGAGCTTCTGCTAATCTTGCTGCTACTAAAATGTCAGAGTGTGTAC
TTGGACAATCAAAAAGAGTTGATTTTTGTGGAAAGGGCTATCATCTTATG
TCCTTCCCTCAGTCAGCACCTCATGGTGTAGTCTTCTTGCATGTGACTTA
TGTCCCTGCACAAGAAAAGAACTTCACAACTGCTCCTGCCATTTGTCATG
ATGGAAAAGCACACTTTCCTCGTGAAGGTGTCTTTGTTTCAAATGGCACA
CACTGGTTTGTAACACAAAGGAATTTTTATGAACCACAAATCATTACTAC
AGACAACACATTTGTGTCTGGTAACTGTGATGTTGTAATAGGAATTGTCA
ACAACACAGTTTATGATCCTTTGCAACCTGAATTAGACTCATTCAAGGAG
GAGTTAGATAAATATTTTAAGAATCATACATCACCAGATGTTGATTTAGG
TGACATCTCTGGCATTAATGCTTCAGTTGTAAACATTCAAAAAGAAATTG
ACCGCCTCAATGAGGTTGCCAAGAATTTAAATGAATCTCTCATCGATCTC
CAAGAACTTGGAAAGTATGAGCAGTATATAAAATGGCCATGGTACATTTG
GCTAGGTTTTATAGCTGGCTTGATTGCCATAGTAATGGTGACAATTATGC
TTTGCTGTATGACCAGTTGCTGTAGTTGTCTCAAGGGCTGTTGTTCTTGT
GGATCCTGCTGCAAATTTGATGAAGACGACTCTGAGCCAGTGCTCAAAGG
AGTCAAATTACATTACACATAA

The DNA sequence of the exemplary SARS-CoV-2 S protomer provided above can be modified to introduce the amino acid substitutions and deletions disclosed herein for prefusion stabilization.

In several embodiments, the nucleic acid molecule encodes a precursor of the protomer, that, when expressed in an appropriate cell, is processed into a disclosed SARS-CoV-2 S ectodomain protomer that can self-assemble into the corresponding recombinant SARS-CoV-2 S ectodomain trimer. For example, the nucleic acid molecule can encode a recombinant SARS-CoV-2 S ectodomain including a N-terminal signal sequence for entry into the cellular secretory system that is proteolytically cleaved in the during processing of the recombinant SARS-CoV-2 S ectodomain in the cell.

In several embodiments, the nucleic acid molecule encodes a precursor SARS-CoV-2 S polypeptide that, when expressed in an appropriate cell, is processed into a disclosed recombinant SARS-CoV-2 S ectodomain protomer including S1 and S2 polypeptides, wherein the recombinant SARS-CoV-2 S ectodomain protomer includes the stabilizing modifications described herein, and optionally can be linked to a trimerization domain, such as a T4 Fibritin trimerization domain.

Exemplary nucleic acids can be prepared by cloning techniques. Examples of appropriate cloning and sequencing techniques, and instructions sufficient to direct persons of skill through many cloning exercises are known (see, e.g., Sambrook et al. (Molecular Cloning: A Laboratory Manual, 4^{th} ed, Cold Spring Harbor, N.Y., 2012) and Ausubel et al. (In Current Protocols in Molecular Biology, John Wiley & Sons, New York, through supplement 104, 2013).

Nucleic acids can also be prepared by amplification methods Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

The polynucleotides encoding a disclosed recombinant SARS-CoV-2 S ectodomain protomer can include a recombinant DNA which is incorporated into a vector (such as an expression vector) into an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single and double forms of DNA.

Polynucleotide sequences encoding a disclosed recombinant SARS-CoV-2 S ectodomain protomer can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

DNA sequences encoding the disclosed recombinant S ectodomain protomer can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Hosts can include microbial, yeast, insect and mammalian organisms. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, e.g., Helgason and Miller (Eds.), 2012, Basic Cell Culture Protocols (Methods in Molecular Biology), 4^{th} Ed., Humana Press). Examples of commonly used mammalian host cell lines are VERO and HeLa cells, CHO cells, and WI38, BHK, and COS cell lines, although cell lines may be used, such as cells designed to provide higher expression, desirable glycosylation patterns, or other features. In some embodiments, the host cells include HEK293 cells or derivatives thereof, such as GnTI^{-/-} cells (ATCC® No. CRL-3022), or HEK-293F cells.

Transformation of a host cell with recombinant DNA can be carried out by conventional techniques. Where the host is prokaryotic, such as, but not limited to, E. coli, competent cells which are capable of DNA uptake can be prepared from cells harvested after exponential growth phase and subsequently treated by the CaCl₂ method using standard procedures. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or viral vectors can be used. Eukaryotic cells can also be co-transformed with polynucleotide sequences encoding a disclosed antigen, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Viral Expression Vectors, Springer press, Muzyczka ed., 2011). Appropriate expression systems such as plasmids and vectors of use in producing proteins in cells including higher eukaryotic cells such as the COS, CHO, HeLa and myeloma cell lines.

In one non-limiting example, a disclosed immunogen is expressed using the pVRC8400 vector (described in Barouch et al., J. Virol., 79, 8828-8834, 2005, which is incorporated by reference herein).

Modifications can be made to a nucleic acid encoding a disclosed recombinant SARS-CoV-2 S ectodomain protomer without diminishing its biological activity. Some modifications can be made to facilitate the cloning, expression, or incorporation of the targeting molecule into a fusion protein. Such modifications are well known to those of skill in the art and include, for example, termination codons, a methionine added at the amino terminus to provide an initiation, site, additional amino acids placed on either terminus to create conveniently located restriction sites, or additional amino acids (such as poly His) to aid in purification steps.

In some embodiments, the disclosed recombinant SARS-CoV-2 S ectodomain protomer can be expressed in cells under conditions where the recombinant SARS-CoV-2 S ectodomain protomer can self-assemble into trimers which are secreted from the cells into the cell media. In such embodiments, each recombinant SARS-CoV-2 S ectodomain protomer contains a leader sequence (signal peptide) that causes the protein to enter the secretory system, where the signal peptide is cleaved and the protomers form a trimer, before being secreted in the cell media. The medium can be centrifuged and recombinant SARS-CoV-2 S ectodomain trimer purified from the supernatant.

### IV. VIRAL VECTORS

A nucleic acid molecule encoding a protomer of a disclosed recombinant SARS-CoV-2 S ectodomain trimer can be included in a viral vector, for example, for expression of the immunogen in a host cell, or for immunization of a subject as disclosed herein. In some embodiments, the viral vectors are administered to a subject as part of a prime-boost vaccination. In several embodiments, the viral vectors are included in a vaccine, such as a primer vaccine or a booster vaccine for use in a prime-boost vaccination.

In several examples, the viral vector can be replication-competent. For example, the viral vector can have a mutation in the viral genome that does not inhibit viral replication in host cells. The viral vector also can be conditionally replication-competent. In other examples, the viral vector is replication-deficient in host cells.

A number of viral vectors have been constructed, that can be used to express the disclosed antigens, including polyoma, i.e., SV40 (Madzak et al., 1992, J. Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Natl. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

In several embodiments, the viral vector can include an adenoviral vector that expresses a protomer of a disclosed recombinant SARS-CoV-2 S ectodomain trimer. Adenovirus from various origins, subtypes, or mixture of subtypes can be used as the source of the viral genome for the adenoviral vector. Non-human adenovirus (e.g., simian, chimpanzee, gorilla, avian, canine, ovine, or bovine adenoviruses) can be used to generate the adenoviral vector. For example, a simian adenovirus can be used as the source of the viral genome of the adenoviral vector. A simian adenovirus can be of serotype 1, 3, 7, 11, 16, 18, 19, 20, 27, 33, 38, 39, 48, 49, 50, or any other simian adenoviral serotype. A simian adenovirus can be referred to by using any suitable abbreviation known in the art, such as, for example, SV, SAdV, SAV or sAV. In some examples, a simian adenoviral vector is a simian adenoviral vector of serotype 3, 7, 11, 16, 18, 19, 20, 27, 33, 38, or 39. In one example, a chimpanzee serotype C Ad3 vector is used (see, e.g., Peruzzi et al., Vaccine, 27:1293-1300, 2009). Human adenovirus can be used as the source of the viral genome for the adenoviral vector. Human adenovirus can be of various subgroups or serotypes. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22, 23, 24, 25, 26, 27, 28, 29, 30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. The person of ordinary skill in the art is familiar with replication competent and deficient adenoviral vectors (including singly and multiply replication deficient adenoviral vectors). Examples of replication-deficient adenoviral vectors, including multiply replication-deficient adenoviral vectors, are disclosed in U.S. Pat. Nos. 5,837,511; 5,851,806; 5,994,106; 6,127,175; 6,482,616; and 7,195,896, and International Patent Application Nos. WO 94/28152, WO 95/02697, WO 95/16772, WO 95/34671, WO 96/22378, WO 97/12986, WO 97/21826, and WO 03/022311.

### V. VIRUS-LIKE PARTICLES

In some embodiments, a virus-like particle (VLP) is provided that includes a disclosed recombinant SARS-CoV-2 S ectodomain trimer. Typically such VLPs include a recombinant SARS-CoV-2 S ectodomain trimer that is membrane anchored by a C-terminal transmembrane domain, for example the recombinant SARS-CoV-2 S ectodomain protomers in the trimer each can be linked to a transmembrane domain and cytosolic tail from SARS-CoV-2 S protein. VLPs lack the viral components that are required for virus replication and thus represent a highly attenuated, replication-incompetent form of a virus. However, the VLP can display a polypeptide (e.g., a recombinant SARS-CoV-2 S ectodomain trimer) that is analogous to that expressed on infectious virus particles and can eliciting an immune response to SARS-CoV-2 when administered to a subject. Virus like particles and methods of their production are known and familiar to the person of ordinary skill in the art, and viral proteins from several viruses are known to form VLPs, including human papillomavirus, HIV (Kang et al., Biol. Chem. 380: 353-64 (1999)), Semliki-Forest virus (Notka et al., Biol. Chem. 380: 341-52 (1999)), human polyomavirus (Goldmann et al., J. Virol. 73: 4465-9 (1999)), rotavirus (Jiang et al., Vaccine 17: 1005-13 (1999)), parvovirus (Casal, Biotechnology and Applied Biochemistry, Vol 29, Part 2, pp 141-150 (1999)), canine parvovirus (Hurtado et al., J. Virol. 70: 5422-9 (1996)), hepatitis E virus (Li et al., J. Virol. 71: 7207-13 (1997)), and Newcastle disease virus. The formation of such VLPs can be detected by any suitable technique. Examples of suitable techniques known in the art for detection of VLPs in a medium include, e.g., electron microscopy techniques, dynamic light scattering (DLS), selective chromatographic separation (e.g., ion exchange, hydrophobic interaction, and/or size exclusion chromatographic separation of the VLPs) and density gradient centrifugation.

### VI. IMMUNOGENIC COMPOSITIONS

Immunogenic compositions comprising a disclosed immunogen (e.g., a disclosed recombinant SARS-CoV-2 S ectodomain trimer or nucleic acid molecule encoding a protomer of disclosed recombinant SARS-CoV-2 S ectodomain trimer) and a pharmaceutically acceptable carrier are also provided. Such pharmaceutical compositions can be administered to subjects by a variety of administration modes known to the person of ordinary skill in the art, for example, intramuscular, intradermal, subcutaneous, intravenous, intra-arterial, intra-articular, intraperitoneal, intranasal, sublingual, tonsillar, oropharyngeal, or other parenteral and mucosal routes. Actual methods for preparing administrable compositions will be known or apparent to those skilled in the art and are described in more detail in such publications as Remingtons Pharmaceutical Sciences, 19^{th} Ed., Mack Publishing Company, Easton, Pa., 1995.

Thus, an immunogen described herein can be formulated with pharmaceutically acceptable carriers to help retain biological activity while also promoting increased stability during storage within an acceptable temperature range. Potential carriers include, but are not limited to, physiologically balanced culture medium, phosphate buffer saline solution, water, emulsions (e.g., oil/water or water/oil emulsions), various types of wetting agents, cryoprotective additives or stabilizers such as proteins, peptides or hydrolysates (e.g., albumin, gelatin), sugars (e.g., sucrose, lactose, sorbitol), amino acids (e.g., sodium glutamate), or other protective agents. The resulting aqueous solutions may be packaged for use as is or lyophilized. Lyophilized preparations are combined with a sterile solution prior to administration for either single or multiple dosing.

Formulated compositions, especially liquid formulations, may contain a bacteriostat to prevent or minimize degradation during storage, including but not limited to effective concentrations (usually ≤1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The immunogenic compositions of the disclosure can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate.

The immunogenic composition may optionally include an adjuvant to enhance an immune response of the host. Suitable adjuvants are, for example, toll-like receptor agonists, alum, AlPO4, alhydrogel, Lipid-A and derivatives or variants thereof, oil-emulsions, saponins, neutral liposomes, liposomes containing the vaccine and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, Ind.) and IL-12 (Genetics Institute, Cambridge, Mass.), among many other suitable adjuvants well known in the art, may be used as an adjuvant (Newman et al., 1998, Critical Reviews in Therapeutic Drug Carrier Systems 15:89-142). These adjuvants have the advantage in that they help to stimulate the immune system in a non-specific way, thus enhancing the immune response to a pharmaceutical product.

In some instances it may be desirable to combine a disclosed immunogen with other pharmaceutical products (e.g., vaccines) which induce protective responses to other agents. For example, a composition including a recombinant SARS-CoV-2 S ectodomain trimer as described herein can be can be administered simultaneously or sequentially with other vaccines recommended by the Advisory Committee on Immunization Practices (ACIP; cdc.gov/vaccines/acip/index.html) for the targeted age group (e.g., infants from approximately one to six months of age), such as an influenza vaccine or a varicella zoster vaccine. As such, a disclosed immunogen including a recombinant SARS-CoV-2 S ectodomain trimer described herein may be administered simultaneously or sequentially with vaccines against, for example, hepatitis B (HepB), diphtheria, tetanus and pertussis (DTaP), pneumococcal bacteria (PCV), Haemophilus influenzae type b (Hib), polio, influenza and rotavirus.

In some embodiments, the composition can be provided as a sterile composition. The pharmaceutical composition typically contains an effective amount of a disclosed immunogen and can be prepared by conventional techniques. Typically, the amount of immunogen in each dose of the immunogenic composition is selected as an amount which induces an immune response without significant, adverse side effects. In some embodiments, the composition can be provided in unit dosage form for use to induce an immune response in a subject. A unit dosage form contains a suitable single preselected dosage for administration to a subject, or suitable marked or measured multiples of two or more preselected unit dosages, and/or a metering mechanism for administering the unit dose or multiples thereof. In other embodiments, the composition further includes an adjuvant.

### VII. METHODS OF INDUCING AN IMMUNE RESPONSE

The disclosed immunogens (e.g., recombinant SARS-CoV-2 S ectodomain trimer, a nucleic acid molecule (such as an RNA molecule) or vector encoding a protomer of a disclosed recombinant SARS-CoV-2 S ectodomain trimer, or a protein nanoparticle or virus like particle comprising a disclosed recombinant SARS-CoV-2 S ectodomain trimer) can be administered to a subject to induce an immune response to SARS-CoV-2 S protein in the subject. In a particular example, the subject is a human. The immune response can be a protective immune response, for example a response that inhibits subsequent infection with SARS-CoV-2. Elicitation of the immune response can also be used to treat or inhibit SARS-CoV-2 infection and illnesses associated with the SARS-CoV-2 infection.

A subject can be selected for immunization that has or is at risk for developing SARS-CoV-2 infection, for example because of exposure or the possibility of exposure to the SARS-CoV-2. Following administration of a disclosed immunogen, the subject can be monitored for infection or symptoms associated with SARS-CoV-2 infection.

Typical subjects intended for immunization with the immunogens and methods of the present disclosure include humans, as well as non-human primates and other animals. To identify subjects for immunization according to the methods of the disclosure, accepted screening methods are employed to determine risk factors associated with a targeted or suspected disease or condition, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods to detect and/or characterize coronavirus infection. These and other routine methods allow the clinician to select patients in need of immunization using the methods and pharmaceutical compositions of the disclosure.

The administration of a disclosed immunogen can be for prophylactic or therapeutic purpose. When provided prophylactically, the immunogen is provided in advance of any symptom, for example, in advance of infection. The prophylactic administration of the immunogen serves to prevent or ameliorate the course of any subsequent infection. When provided therapeutically, the immunogen is provided at or after the onset of a symptom of infection, for example, after development of a symptom of SARS-CoV-2 infection or after diagnosis with the SARS-CoV-2 infection. The immunogen can thus be provided prior to the anticipated exposure to the SARS-CoV-2 so as to attenuate the anticipated severity, duration or extent of an infection and/or associated disease symptoms, after exposure or suspected exposure to the SARS-CoV-2, or after the actual initiation of an infection.

The immunogens described herein, and immunogenic compositions thereof, are provided to a subject in an amount effective to induce or enhance an immune response against the SARS-CoV-2 S protein in the subject, preferably a human. The actual dosage of disclosed immunogen will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimum prophylactic or therapeutic response.

An immunogenic composition including one or more of the disclosed immunogens can be used in coordinate (or prime-boost) vaccination protocols or combinatorial formulations. In certain embodiments, novel combinatorial immunogenic compositions and coordinate immunization protocols employ separate immunogens or formulations, each directed toward eliciting an anti-viral immune response, such as an immune response to SARS-CoV-2 S protein. Separate immunogenic compositions that elicit the anti-viral immune response can be combined in a polyvalent immunogenic composition administered to a subject in a single immunization step, or they can be administered separately (in monovalent immunogenic compositions) in a coordinate (or prime-boost) immunization protocol.

There can be several boosts, and each boost can be a different disclosed immunogen. In some examples that the boost may be the same immunogen as another boost, or the prime. The prime and boost can be administered as a single dose or multiple doses, for example two doses, three doses, four doses, five doses, six doses or more can be administered to a subject over days, weeks or months. Multiple boosts can also be given, such one to five (e.g., 1, 2, 3, 4 or 5 boosts), or more. Different dosages can be used in a series of sequential immunizations. For example a relatively large dose in a primary immunization and then a boost with relatively smaller doses.

In some embodiments, the boost can be administered about two, about three to eight, or about four, weeks following the prime, or about several months after the prime. In some embodiments, the boost can be administered about 5, about 6, about 7, about 8, about 10, about 12, about 18, about 24, months after the prime, or more or less time after the prime. Periodic additional boosts can also be used at appropriate time points to enhance the subject's “immune memory.” The adequacy of the vaccination parameters chosen, e.g., formulation, dose, regimen and the like, can be determined by taking aliquots of serum from the subject and assaying antibody titers during the course of the immunization program. In addition, the clinical condition of the subject can be monitored for the desired effect, e.g., prevention of infection or improvement in disease state (e.g., reduction in viral load). If such monitoring indicates that vaccination is sub-optimal, the subject can be boosted with an additional dose of immunogenic composition, and the vaccination parameters can be modified in a fashion expected to potentiate the immune response.

In some embodiments, the prime-boost method can include DNA-primer and protein-boost vaccination protocol to a subject. The method can include two or more administrations of the nucleic acid molecule or the protein.

For protein therapeutics, typically, each human dose will comprise 1-1000 μg of protein, such as from about 1 μg to about 100 μg, for example, from about 1 μg to about 50 μg, such as about 1 μg, about 2 μg, about 5 μg, about 10 μg, about 15 μg, about 20 μg, about 25 μg, about 30 μg, about 40 μg, or about 50 μg.

The amount utilized in an immunogenic composition is selected based on the subject population (e.g., infant or elderly). An optimal amount for a particular composition can be ascertained by standard studies involving observation of antibody titers and other responses in subjects. It is understood that a effective amount of a disclosed immunogen, such as a disclosed recombinant SARS-CoV-2 S ectodomain trimer, viral vector, or nucleic acid molecule, in a immunogenic composition, can include an amount that is ineffective at eliciting an immune response by administration of a single dose, but that is effective upon administration of multiple dosages, for example in a prime-boost administration protocol.

Upon administration of an immunogen of this disclosure, the immune system of the subject typically responds by producing antibodies specific for the SARS-CoV-2 S ectodomain trimer included in the immunogen. Such a response signifies that an immunologically effective dose was delivered to the subject.

In some embodiments, the antibody response of a subject will be determined in the context of evaluating effective dosages/immunization protocols. In most instances it will be sufficient to assess the antibody titer in serum or plasma obtained from the subject. Decisions as to whether to administer booster inoculations and/or to change the amount of the therapeutic agent administered to the individual can be at least partially based on the antibody titer level. The antibody titer level can be based on, for example, an immunobinding assay which measures the concentration of antibodies in the serum which bind to an antigen including, for example, the recombinant SARS-CoV-2 S ectodomain trimer included in the immunogen.

SARS-CoV-2 infection does not need to be completely eliminated or reduced or prevented for the methods to be effective. For example, elicitation of an immune response to SARS-CoV-2 with one or more of the disclosed immunogens can reduce or inhibit SARS-CoV-2 infection by a desired amount, for example, by at least 10%, at least 20%, at least 50%, at least 60%, at least 70%, at least 80%, at least 90%, at least 95%, at least 98%, or even at least 100% (elimination or prevention of detectable infected cells), as compared to SARS-CoV-2 infection in the absence of the immunogen. In additional examples, SARS-CoV-2 replication can be reduced or inhibited by the disclosed methods. SARS-CoV-2 replication does not need to be completely eliminated for the method to be effective. For example, the immune response elicited using one or more of the disclosed immunogens can reduce SARS-CoV-2 replication by a desired amount, for example, by at least 10%, at least 20%, at least 50%, at least 60%, at least 70%, at least 80%, at least 90%, at least 95%, at least 98%, or even at least 100% (elimination or prevention of detectable SARS-CoV-2 replication, as compared to SARS-CoV-2 replication in the absence of the immune response.

In some embodiments, the disclosed immunogen is administered to the subject simultaneously with the administration of the adjuvant. In other embodiments, the disclosed immunogen is administered to the subject after the administration of the adjuvant and within a sufficient amount of time to induce the immune response.

One approach to administration of nucleic acids is direct immunization with plasmid DNA, such as with a mammalian expression plasmid Immunization by nucleic acid constructs is well known in the art and taught, for example, in U.S. Pat. No. 5,643,578 (which describes methods of immunizing vertebrates by introducing DNA encoding a desired antigen to elicit a cell-mediated or a humoral response), and U.S. Pat. Nos. 5,593,972 and 5,817,637 (which describe operably linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids (or of the synthetic peptides themselves), and immune-stimulating constructs, or ISCOMS™, negatively charged cage-like structures of 30-40 nm in size formed spontaneously on mixing cholesterol and Quil A™ (saponin). Protective immunity has been generated in a variety of experimental models of infection, including toxoplasmosis and Epstein-Barr virus-induced tumors, using ISCOMS™ as the delivery vehicle for antigens (Mowat and Donachie, Immunol. Today 12:383, 1991). Doses of antigen as low as 1 μg encapsulated in ISCOMS™ have been found to produce Class I mediated CTL responses (Takahashi et al., Nature 344:873, 1990).

In some embodiments, a plasmid DNA vaccine is used to express a disclosed immunogen in a subject. For example, a nucleic acid molecule encoding a disclosed immunogen can be administered to a subject to induce an immune response to the SARS-CoV-2 S protein included in the immunogen. In some embodiments, the nucleic acid molecule can be included on a plasmid vector for DNA immunization, such as the pVRC8400 vector (described in Barouch et al., J. Virol, 79, 8828-8834, 2005, which is incorporated by reference herein).

In another approach to using nucleic acids for immunization, a disclosed recombinant SARS-CoV-2 S ectodomain or recombinant SARS-CoV-2 S ectodomain trimer can be expressed by attenuated viral hosts or vectors or bacterial vectors. Recombinant vaccinia virus, adeno-associated virus (AAV), herpes virus, retrovirus, cytomegalo virus or other viral vectors can be used to express the peptide or protein, thereby eliciting a CTL response. For example, vaccinia vectors and methods useful in immunization protocols are described in U.S. Pat. No. 4,722,848. BCG (Bacillus Calmette Guerin) provides another vector for expression of the peptides (see Stover, Nature 351:456-460, 1991).

In one embodiment, a nucleic acid encoding a disclosed recombinant SARS-CoV-2 S ectodomain or SARS-CoV-2 S ectodomain trimer is introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

In another embodiment, an mRNA-based immunization protocol can be used to deliver a nucleic acid encoding a disclosed recombinant SARS-CoV-2 S ectodomain directly into cells. In some embodiments, nucleic acid-based vaccines based on mRNA may provide a potent alternative to the previously mentioned approaches. mRNA vaccines preclude safety concerns about DNA integration into the host genome and can be directly translated in the host cell cytoplasm. Moreover, the simple cell-free, in vitro synthesis of RNA avoids the manufacturing complications associated with viral vectors. Two exemplary forms of RNA-based vaccination that can be used to deliver a nucleic acid encoding a disclosed recombinant SARS-CoV-2 S ectodomain include conventional non-amplifying mRNA immunization (see, e.g., Petsch et al., “Protective efficacy of in vitro synthesized, specific mRNA vaccines against influenza A virus infection,” Nature biotechnology, 30(12):1210-6, 2012) and self-amplifying mRNA immunization (see, e.g., Geall et al., “Nonviral delivery of self-amplifying RNA vaccines,” PNAS, 109(36): 14604-14609, 2012; Magini et al., “Self-Amplifying mRNA Vaccines Expressing Multiple Conserved Influenza Antigens Confer Protection against Homologous and Heterosubtypic Viral Challenge,” PLoS One, 11(8):e0161193, 2016; and Brito et al., “Self-amplifying mRNA vaccines,” Adv Genet., 89:179-233, 2015).

Administration of an effective amount of one or more of the disclosed immunogens to a subject induces a neutralizing immune response in the subject. To assess neutralization activity, following immunization of a subject, serum can be collected from the subject at appropriate time points, frozen, and stored for neutralization testing. Methods to assay for neutralization activity include, but are not limited to, plaque reduction neutralization (PRNT) assays, microneutralization assays, flow cytometry based assays, single-cycle infection assays. In some embodiments, the serum neutralization activity can be assayed using a SARS-CoV-2 pseudovirus, similar to that used for SARS-CoV (Martin et al., Vaccine 26, 6338, 2008; Yang et al., Nature 428, 561, 2004; Naldini et al., PNAS 93, 11382, 1996; Yang et al., PNAS 102, 797, 2005).

### EXAMPLES

The following examples are provided to illustrate particular features of certain embodiments, but the scope of the claims should not be limited to those features exemplified.

### Example 1

### Prefusion Stabilized SARS-CoV-2 S Protein

This example describes development of a recombinant SARS-CoV-2 S ectodomain trimer that is stabilized in a prefusion conformation.

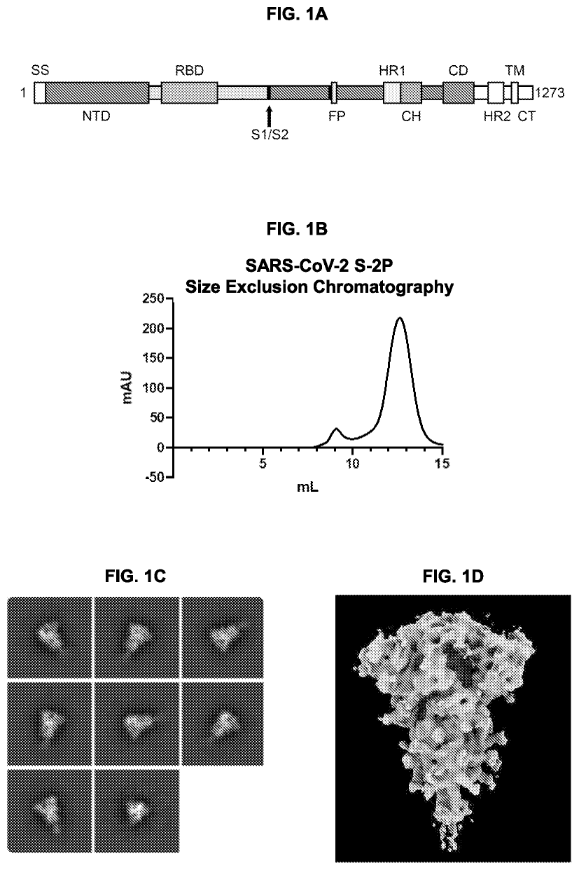

The sequence of the SARS-CoV-2 S protein was investigated to reveal details about its architecture. From this, the possibility of using amino acid substitutions to stabilize the S protein in its prefusion conformation was assessed. Two mutations were identified to be particularly effective for stabilizing the SARS-CoV-2 S protein in its prefusion conformation: K986P and V987P. SARS-CoV-2 S with K986P and V987P substitutions is referred to as “S-2P.” These two proline substitutions are located at the top portion (membrane distal) of the SARS-CoV-2 S2, between the central helix and HR1, and prevent pre-to-postfusion conformational changes. FIG. 1A shows a schematic diagram of SARS-CoV-2 S domains.

The prefusion SARS-CoV-2 S protein (with K986P and V987P) was expressed as a soluble protein (without TM and CT) with a C-terminal T4 fibritin trimerization domain. Including the signal peptide and T4 Fibritin trimerization domain, a protomer sequence of the SARS-CoV-2 S trimer with the K986P and V987P substitutions are provided as SEQ ID NO: 2. C-terminal to the trimerization domain, the expressed protein included purification and detection tags including an HRV3C cleavage site, a 6×His-tag and a Twin-Strep-tag. Following sequence verification, expression plasmids were transiently transfected into FreeStyle293 cells. Cultures were harvested six days later, and secreted protein was purified from the supernatant by passage over Ni²⁺-NTA and StrepTactin resin using the affinity tags on the C-terminus of the proteins. The purified proteins were then be passed over a size-exclusion column to assess their oligomeric state (FIG. 1B) and to isolate monodisperse fractions corresponding to trimeric ectodomains. Protein expression levels were then assessed by SDS-PAGE.

Prefusion stabilization of the SARS-CoV-2 S protein is preliminarily indicated by increased expression levels when these mutations are combined compared to a corresponding wild-type protein.

The conformation of the double proline mutant SARS-CoV-2 S variant was assessed by negative stain electron microscopy (FIGS. 1C and 1D). The S variant with the double proline mutations was homogeneous and formed trimers in the expected prefusion shape. Each of these ectodomain trimers was purified as a single peak and formed trimers in the typical prefusion conformation. In contrast, corresponding S proteins with native sequences formed trimers of mixed conformation, with some trimers in the typical prefusion conformation and others in the typical elongated post-fusion conformation.

### Example 2

### Prefusion Stabilized SARS-CoV-2 S Protein Elicits a Neutralizing Immune Response in an Animal Model

This example describes elicitation of a neutralizing immune response to SARS-CoV-2 infection in an animal model using a prefusion-stabilized SARS-CoV-2 S protein as an immunogen.

Soluble prefusion-stabilized SARS-CoV-2 S protein was prepared as described in Example 1. As a control, SARS-CoV-2 S without the K986P and V987P substitutions was expressed as a soluble protein (without TM and CT) with a C-terminal T4 fibritin trimerization domain. SARS-CoV2 S sequence without engineered substitutions is referred to as SARS-CoV-2 S WT.

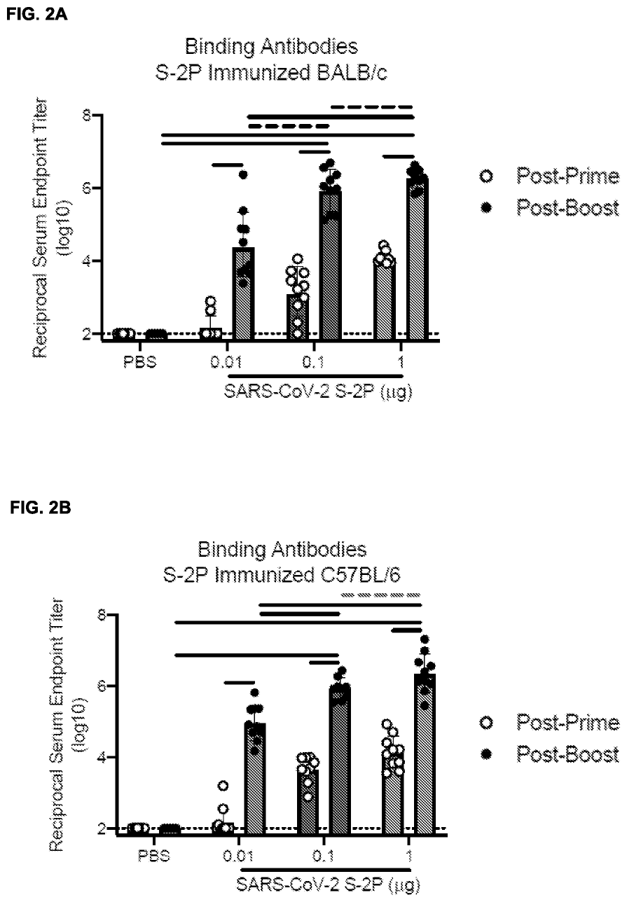

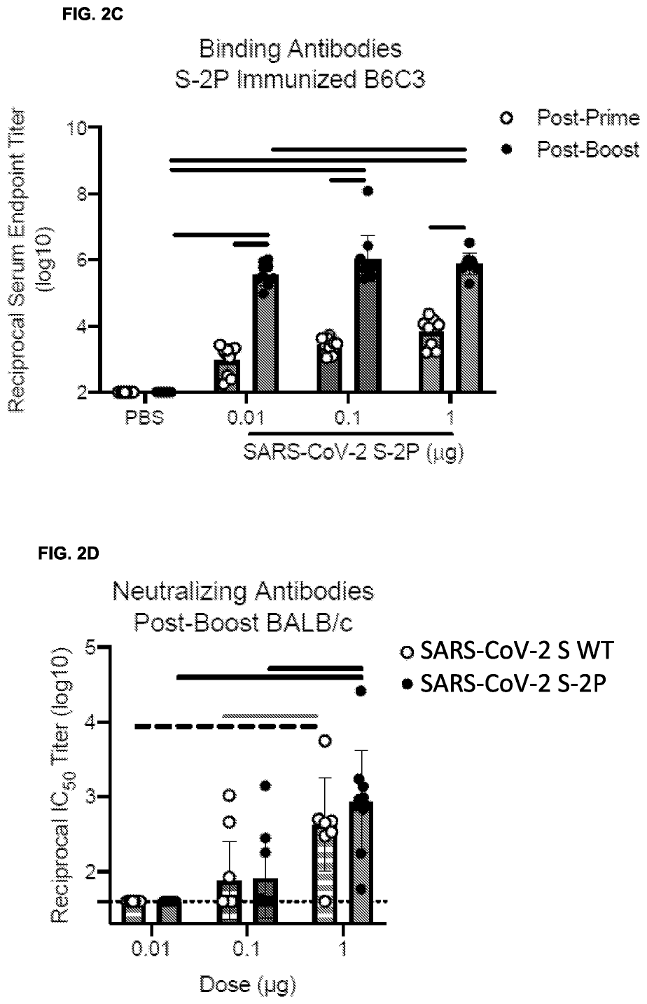

Three different mouse strains, BALB/cJ, C57BL/6J, and B6C3F1/J mice were immunized at weeks 0 and 3 with PBS, 0.01 μg, 0.1 μg, or 1 μg of the soluble SARS-CoV-2 S WT or soluble SARS-CoV-2 S-2P adjuvanted with Sigma Adjuvant System (SAS), and sera were collected two weeks post-prime and two weeks post-boost. Sera from SARS-CoV-2 S-2P immunized mice were assessed for SARS-CoV-2 S-specific IgG by ELISA (FIGS. 2A-2C). Post-boost sera from both S WT and S-2P-immunized BALB/cJ mice were assessed for neutralizing antibodies against homotypic SARS-CoV-2 pseudovirus (FIG. 2D). The results show that soluble SARS-CoV-2 S-2P elicits dose-dependent S-specific binding antibodies after the prime and boost conditions, and that 1 μg of the soluble SARS-CoV-2 S-2P immunogen elicited a robust neutralizing antibody response in an animal model.

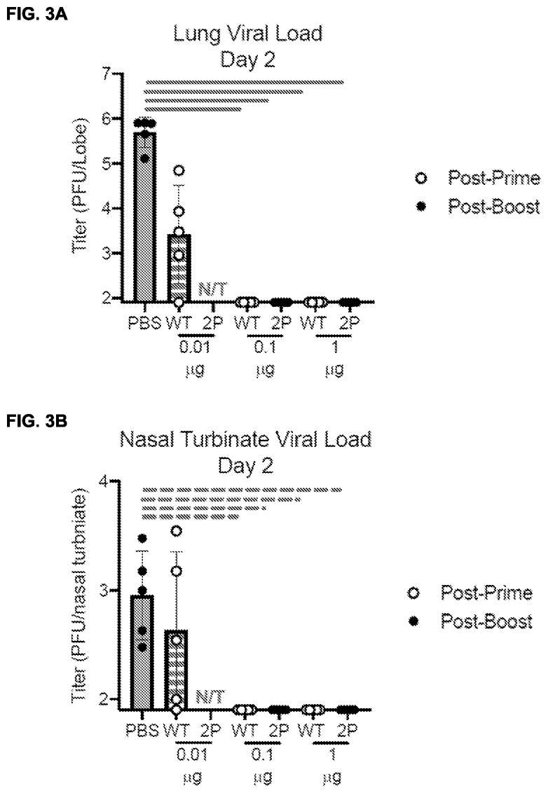

The ability of soluble SARS-CoV-2 S WT and soluble SARS-CoV-2 S-2P immunization to protect mice against viral replication was assessed. BALB/cJ mice were immunized at weeks 0 and 3 with PBS, 0.01 μg, 0.1 μg, or 1 μg of soluble SARS-CoV-2 S WT or soluble SARS-CoV-2 S-2P adjuvanted with SAS. Four weeks post-boost, mice were challenged with mouse-adapted SARS-CoV-2 (described in Dinnon, et al. “a mouse adapted SARS-CoV-2 model for the evaluation of COVID-19 medical countermeasures,” BioRxiv., 2020.05.06.081497, which is incorporated by reference herein). Two days post-challenge, at peak viral load, lungs (FIG. 3A) and nasal turbinates (FIG. 3B) were harvested for assessment of viral load by plaque assay. Groups were compared by one-way AVOVA with multiple comparisons test. The results show that the 0.1 μg and 1 μg conditions eliminated viral replication in upper and lower airways; 0.01 μg S WT did not protect, suggesting this to be the breakthrough dose for S WT. The 0.01 μg S-2P-immunized mice were not challenged (N/T), due to death unrelated to the experiment.

### Example 3

### Protein Nanoparticle Containing Prefusion Stabilized SARS-CoV-2 S Protein as an Immunogen

This example illustrates the prefusion-stabilized SARS-CoV-2 S ectodomain trimer conjugated to a protein nanoparticle scaffold and use thereof as an immunogen.

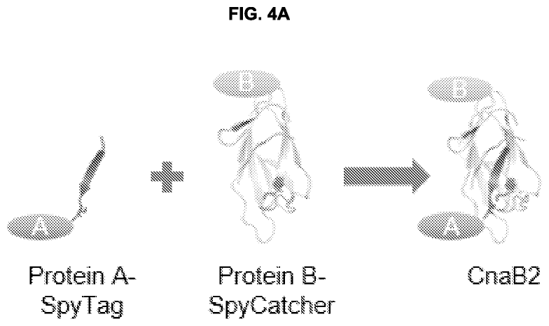

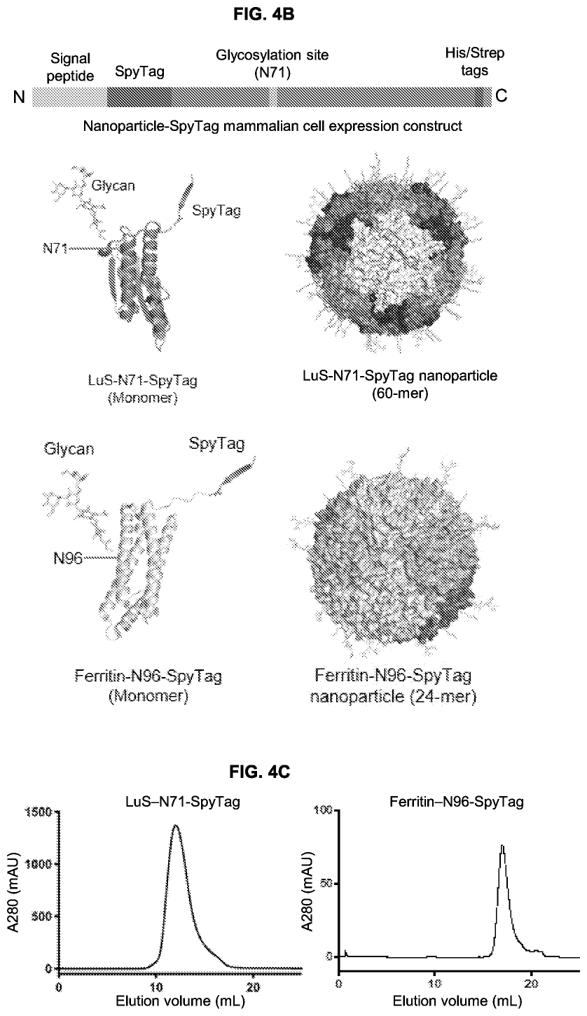

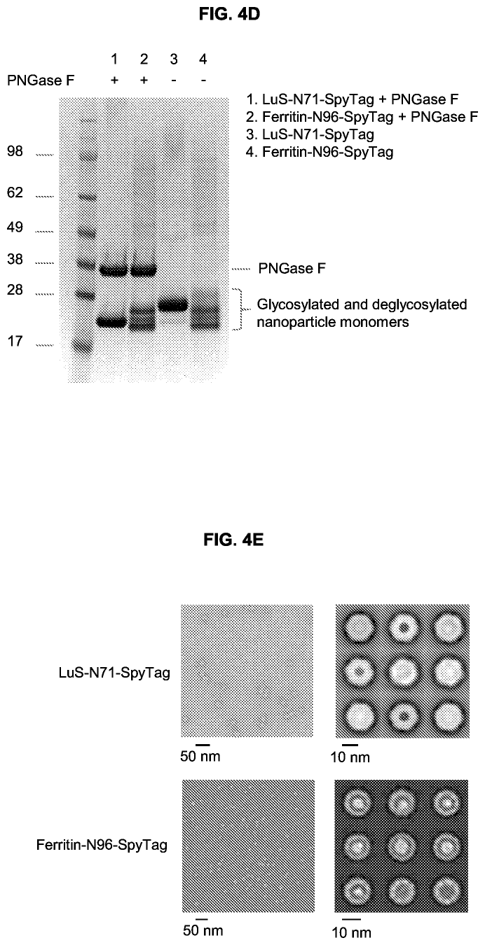

Glycan modification of LuS- and ferritin-nanoparticle scaffolds. To construct a reliable platform for nanoparticle presentation of antigens, Aquifex aeolicus lumazine synthase (LuS) and Helicobacter pylori ferritin were selected as nanoparticle scaffolds, along with the isopeptide bond conjugation system referred to as the SpyTag:SpyCatcher system (Brune, K. D. et al. Plug-and-Display: decoration of Virus-Like Particles via isopeptide bonds for modular immunization. Sci Rep 6, 19234, 2016) to display antigens on nanoparticle surface. The SpyTag:SpyCatcher system is highly specific and stable with an isopeptide bond and has been used for conjugation of antigens on nanoparticle surfaces (FIG. 4A) (See Zakeri, B. et al. “Peptide tag forming a rapid covalent bond to a protein, through engineering a bacterial adhesin.” Proc Natl Acad Sci USA 109, E690-697, (2012); Brune, K. D. et al. Plug-and-Display: decoration of Virus-Like Particles via isopeptide bonds for modular immunization. Sci Rep 6, 19234, (2016)). LuS and ferritin have served as scaffolds for nanoparticle immunogens in clinical studies. The N-terminus of both ferritin and LuS are exposed to the nanoparticle surface and are thus accessible for SpyTag or SpyCatcher attachment (FIG. 4B). The C-terminus of LuS is also accessible on the nanoparticle surface and can be used to display purification tags. Mammalian expression constructs expressing fusion proteins of SpyTag or SpyCatcher with LuS or ferritin were designed. The constructs included both His- and Strep-tags for purification purposes, along with a signal peptide for secretion of the expressed proteins to the medium (FIG. 4B).

Initial constructs yielded low levels of soluble proteins for the nanoparticle-SpyTag or SpyCatcher fusion proteins. To improve protein solubility and expression, glycans were added to the surface of the nanoparticles. A panel of LuS and ferritin constructs with SpyTag and SpyCatcher and added N-linked glycosylation sites was designed (Tables 1 and 2). For LuS constructs, a glycosylation site at position 71 (PDB 1HQK numbering) was added. For ferritin constructs, two potential glycosylation sites (96 and 148) were tested. The addition of N-linked glycosylation sites facilitated expression of soluble nanoparticles in the mammalian cell culture supernatant. Three of the constructs produced superior yields of well-assembled nanoparticles, LuS with N71 and SpyTag at N-terminus (hereafter referred to as LuS-N71-SpyTag), ferritin with N96 and SpyTag, and ferritin S148 (glycan at N146) and SpyTag (Table 1). Of the two ferritin constructs, the ferritin with N96 and SpyTag had a higher yield and was chosen for further studies (referred to as ferritin-N96-SpyTag). Size exclusion chromatography (SEC) and electron microscopy (EM) analyses indicated that LuS-N71-SpyTag formed a homogeneous nanoparticle population in solution (FIGS. 4C and 4E). The ferritin-N96-SpyTag sample comprised mainly of intact nanoparticles with some minor unassembled species (FIGS. 4C and 4D). Negative-stain electron microscopy (EM) images indicated both nanoparticles to be well-assembled with expected sizes (FIG. 4E). Two-dimensional class average revealed more detailed structural features of the nanoparticles, which were consistent with previously published structures of the two nanoparticles. These data indicated the ferritin and LuS nanoparticles were compatible with the SpyTag and glycosylation site addition. These alterations were well tolerated, allowing for robust nanoparticle assembly. To verify the glycosylation of LuS- and ferritin-SpyTag nanoparticles, PNGase F digestion was performed and glycan cleavage was assessed using SDS-PAGE (FIG. 4D). Both nanoparticles showed a band shift in the presence of PNGase F, indicating the presence of N-liked glycan on the nanoparticles and its removal by the amidase digestion. While the glycan cleavage in LuS-N71-SpyTag is distinct, it is less apparent in ferritin-N96-SpyTag, likely due to incomplete glycosylation of ferritin-N96-SpyTag and multiple bands of ferritin on SDS-PAGE. Ferritin has been observed to exhibit a single band on SDS-PAGE in some studies but multiple bands in others, presumably due to protease cleavage at the C terminus or incomplete glycosylation. However, these different sized ferritin molecules assembled correctly as nanoparticles with expected dimensions as indicated by SEC and EM (FIGS. 4C, 4E).

TABLE 1
LuS- and ferritin-nanoparticles with SpyTag.

| | | | Position | Expression |
|---|---|---|---|---|
| | | | of | level |
| Construct ID | SpyTag | SpyCatcher | glycan | (mg/L) |
| Lumazine synthase | | | | |
| LuS-SpyTag no glycan | x | | None | <0.1 |
| LuS-N71-SpyTag* | x | | 71 | 3.0 |
| LuS-C-SpyCatcher no | | x | None | <0.1 |
| glycan | | | | |
| LuS-C71-SpyCatcher | | x | 71 | <0.1 |
| LuS-N-SpyCatcher no | | x | None | <0.1 |
| glycan | | | | |
| LuS-N71-SpyCatcher | | x | 71 | <0.1 |
| Ferritin | | | | |
| Ferritin-SpyTag no glycan | x | | None | <0.1 |
| Ferritin-N96-SpyTag* | x | | 96 | 2.5 |
| Ferritin-S148-SpyTag* | x | | 146 | 1.0 |
| Ferritin-SpyCatcher no | | X | None | <0.1 |
| glycan | | | | |
| Ferritin-N96-SpyCatcher | | X | 96 | <0.1 |
| Ferritin-S148-SpyCatcher | | X | 146 | <0.1 |

TABLE 2
Amino acid sequences of constructs for protein
expression.

| Construct | |
|---|---|
| name | Amino acid sequence |
| LuS-N71- | MDSKGSSQKGSRLLLLLVVSNLLLPQGVVGAHIVM |
| SpyTag | VDAYKPTKGSGSAMQIYEGKLTAEGLRFGIVASRF |
| | NHALVDRLVEGAIDAIVRHGGREEDITLVRVPGSW |
| | EIPVAAGELARKENISAVIAIGVLIRGATPHFDYI |
| | ASEVSKGLADLSLELRKPITFGVITADTLEQAIER |
| | AGTKHGNKGWEAALSAIEMANLFKSLRGGLVPRGS |
| | HHHHHHSAWSHPQFEK |
| | (SEQ ID NO: 12) |
| Ferritin- | MDSKGSSQKGSRLLLLLVVSNLLLPQGVVGQHHHH |
| N96- | HHHHSAWSHPQFEKGGLVPRGGAHIVMVDAYKPTK |
| SpyTag | GGGSGDPMLSKDIIKLLNEQVNKEMQSSNLYMSMS |
| | SWCYTHSLDGAGLFLFDHAAEEYEHAKKLIIFLNE |
| | NNVPVQLTSISAPEHKFEGLTQIFQKAYEHEQNIS |
| | ESINNIVDHAIKSKDHATFNFLQWYVAEQHEEEVL |
| | FKDILDKIELIGNENHGLYLADQYVKGIAKSRKS |
| | (SEQ ID NO: 13) |
| SARS- | MGWSCIILFLVATATGVHSAPELLGGPSVFLFPPK |
| CoV- | PKDTLMISRTPEVTCVVVDVSHEDPEVKFNWYVDG |
| 2 spike- | VEVHNAKTKPREEQYNSTYRVVSVLTVLHQDWLNG |
| Spy- | KEYKCKVSNKALPAPIEKTISKAKGQPREPQVYTL |
| Catcher* | PPSRDELTKNQVSLYCLVKGFYPSDIAVEWESNGQ |
| | PENNYKTTPPVLDSDGSFFLYSKLTVDKSRWQQGN |
| | VFSCSVMHEALHNHYTQKSLSLSPGKGGGGSGGGG |
| | SGGGGSGGGGSAPELLGGPSVFLFPPKPKDTLMIS |
| | RTPEVTCVVVDVSHEDPEVKFNWYVDGVEVHNAKT |
| | KPREEQYNSTYRVVSVLTVLHQDWLNGKEYKCKVS |
| | NKALPAPIEKTISKAKGQPREPQVYTLPPSRDELT |
| | KNQVSLTCLVKGFYPSDIAVEWESNGQPENNYKTT |
| | PPVLDSDGSFFLTSKLTVDKSRWQQGNVFSCSVMH |
| | EALHNHYTQKSLSLSPGKGGSGGGGSGGLEVLFQG |
| | PQCVNLTTRTQLPPAYTNSFTRGVYYPDKVFRSSV |
| | LHSTQDLFLPFFSNVTWFHAIHVSGTNGTKRFDNP |
| | VLPFNDGVYFASTEKSNIIRGWIFGTTLDSKTQSL |
| | LIVNNATNVVIKVCEFQFCNDPFLGVYYHKNNKSW |
| | MESEFRVYSSANNCTFEYVSQPFLMDLEGKQGNFK |
| | NLREFVFKNIDGYFKIYSKHTPINLVRDLPQGFSA |
| | LEPLVDLPIGINITRFQTLLALHRSYLTPGDSSSG |
| | WTAGAAAYYVGYLQPRTFLLKYNENGTITDAVDCA |
| | LDPLSETKCTLKSFTVEKGIYQTSNFRVQPTESIV |
| | RFPNITNLCPFGEVFNATRFASVYAWNRKRISNCV |
| | ADYSVLYNSASFSTFKCYGVSPTKLNDLCFTNVYA |
| | DSFVIRGDEVRQIAPGQTGKIADYNYKLPDDFTGC |
| | VIAWNSNNLDSKVGGNYNYLYRLFRKSNLKPFERD |
| | ISTEIYQAGSTPCNGVEGFNCYFPLQSYGFQPTNG |
| | VGYQPYRVvvLSFELLHAPATVCGPKKSTNLVKNK |
| | CVNFNFNGLTGTGVLTESNKKFLPFQQFGRDIADT |
| | TDAVRDPQTLEILDITPCSFGGVSVITPGTNTSNQ |
| | VAVLYQDVNCTEVPVAIHADQLTPTWRVYSTGSNV |
| | FQTRAGCLIGAEHVNNSYECDIPIGAGICASYQTQ |
| | TNSPGSASSVASQSIIAYTMSLGAENSVAYSNNSI |
| | AIPTNFTISVTTEILPVSMTKTSVDCTMYICGDST |
| | ECSNLLLQYGSFCTQLNRALTGIAVEQDKNTQEVF |
| | AQVKQIYKTPPIKDFGGFNFSQILPDPSKPSKRSF |
| | IEDLLFNKVTLADAGFIKQYGDCLGDIAARDLICA |
| | QKFNGLTVLPPLLTDEMIAQYTSALLAGTITSGWT |
| | FGAGAALQIPFAMQMAYRFNGIGVTQNVLYENQKL |
| | IANQFNSAIGKIQDSLSSTASALGKLQDVVNQNAQ |
| | ALNTLVKQLSSNFGAISSVLNDILSRLDPPEAEVQ |
| | IDRLITGRLQSLQTYVTQQLIRAAEIRASANLAAT |
| | KMSECVLGQSKRVDFCGKGYHLMSFPQSAPHGVVF |
| | LHVTYVPAQEKNFTTAPAICHDGKAHFPREGVFVS |
| | NGTHWFVTQRNFYEPQIITTDNTFVSGNCDVVIGI |
| | VNNTVYDPLQPELDSFKEELDKYFKNHTSPDVDLG |
| | DISGINASVVNIQKEIDRLNEVAKNLNESLIDLQE |
| | LGKYEQGSGYIPEAPRDGQAYVRKDGEWVLLSTFL |
| | GRSGGGLVPQQSGDSATHIKFSKRDEDGKELAGAT |
| | MELRDSSGKTISTWISDGQVKDFYLYPGKYTFVET |
| | AAPDGYEVATAITFTVNEQGQVTVNGKATKGDAHI |
| | (SEQ ID NO: 14) |

*This amino acid sequence includes a single chain Fc purification tag (see reference 38).

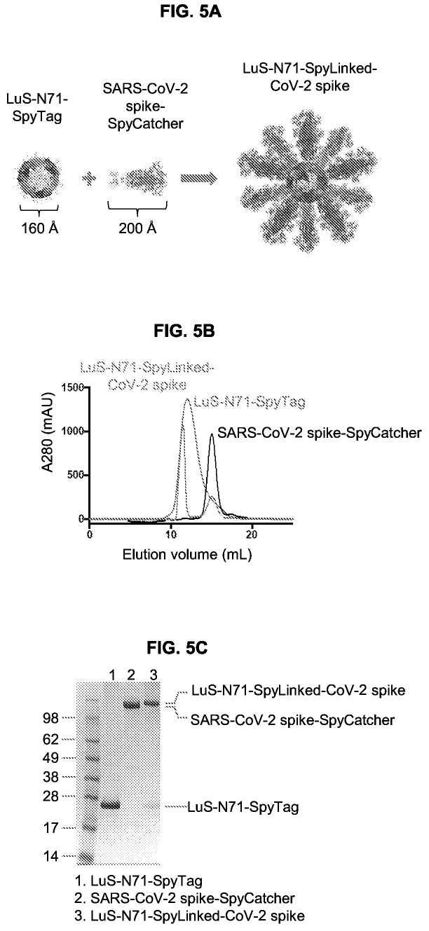

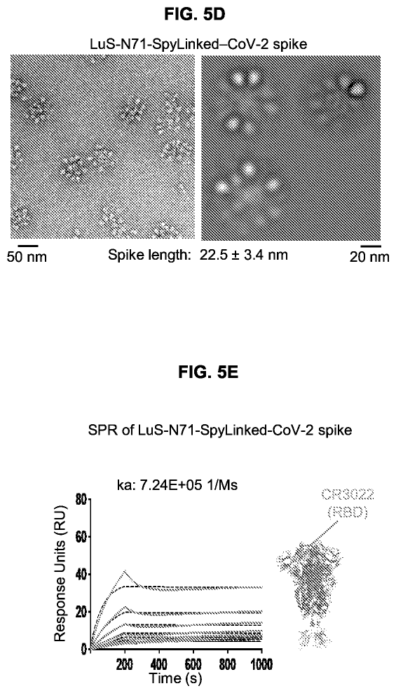

Conjugation of SARS-CoV-2 spike trimer to LuS nanoparticle via SpyTag:SpyCatcher displays the spike trimers homogeneously on the nanoparticle surface. SARS-CoV-2 spike fused with a C-terminal SpyCatcher (SEQ ID NO: 14) was expressed, purified and conjugated to the purified LuS-N71-SpyTag nanoparticle (FIGS. 5A-5C). For this construct, the prefusion SARS-CoV-2 S ectodomain trimer contained protomers including a GSAS substitution to remove the S1/S2 cleavage site, K986P and V987P substitutions for prefusion stabilization and a C-terminal T4 phage fibritin trimerization domain. For purification purposes, the protein included a single-chain Fc tag (for description of the single-chain Fc tag, see Zhou, T. et al. Structure-Based Design with Tag-Based Purification and In-Process Biotinylation Enable Streamlined Development of SARS-CoV-2 Spike Molecular Probes. bioRxiv, 2020.2006.2022.166033). The conjugation mixture was loaded onto an SEC column to purify the conjugated nanoparticle product LuS-N71-SpyLinked-CoV spike from unconjugated LuS-N71-SpyTag and SARS-CoV-2 spike-SpyCatcher (FIG. 5B). SDS-PAGE analysis revealed the conjugated product to have the expected molecular weight, and unconjugated spike-SpyCatcher was not observed after conjugation (FIG. 5C).

To estimate the conjugation efficiency, the intensity of each band on the SDS-PAGE gel image of the conjugated nanoparticle product (FIG. 5C) was measured as a surrogate of mass for each component. Taking into consideration the molecular weight of each component, the molar ratio of each component to total protein in the sample was calculated. Based on this, it is estimated that 91% of all the LuS nanoparticle subunit was conjugated to the spike trimer. Negative stain EM showed LuS-N71-SpyLinked-CoV-2 spike nanoparticle to exhibit the expected size with spike trimers displaying on the LuS nanoparticle surface (FIG. 5D). SPR measurements showed LuS-N71-SpyLinked-SARS-CoV-2 Spike to bind to CR3022 (ter Meulen, J. et al. Human monoclonal antibody combination against SARS coronavirus: synergy and coverage of escape mutants. PLoS Med 3, e237, 2006; Yuan, M. et al. A highly conserved cryptic epitope in the receptor-binding domains of SARS-CoV-2 and SARS-CoV. Science, 2020), an antibody targeting the receptor-binding domain (RBD), indicating successful nanoparticle presentation of the spike trimer using the LuS-SpyTag:SpyCatcher system (FIG. 5E).

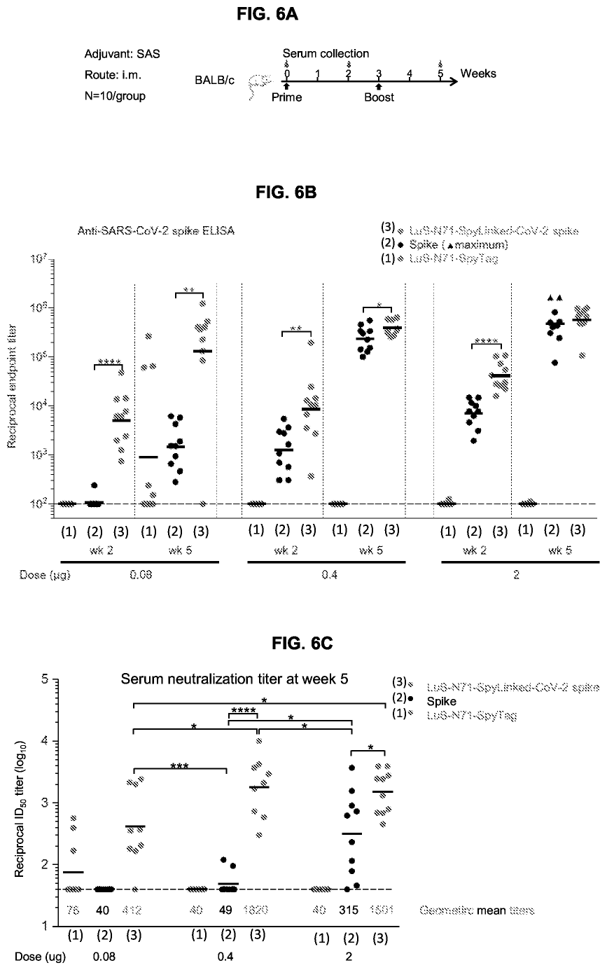

SpyLinked-nanoparticle display increases potential of SARS-CoV-2 spike to elicit neutralizing antibodies. To assess immunogenicity, mice were injected with the LuS-N71-SpyLinked-CoV-2 spike nanoparticle or soluble spike trimers (stabilized by 2P mutation as in Example 1), or mock (LuS-N71-SpyTag) nanoparticles at weeks 0 and 3 (FIG. 6A). Serum samples were collected two weeks after each immunization. After the first immunization, at the lowest immunogen dose of 0.08 μg, spike nanoparticle-immune sera exhibited an anti-SARS-CoV-2 spike ELISA geometric mean titer of 5,116, whereas only 1 out of 10 trimeric spike-immunized sera exhibited a measurable titer (FIG. 6B); after a second immunization, titers for the spike nanoparticle-immune sera increased substantially, by approximately 25-fold Immunizations with higher doses of spike nanoparticle (0.4 and 2.0 μg) increased titers more incrementally, both at week 2 and at week 5. By contrast, increases in dose of the spike trimer raised ELISA titers more substantially, with two of the mice in the 2.0 μg spike-trimer immune sera reaching the assay upper limit of detection with a titer of 1,638,400 (FIG. 6B).

Further, pseudovirus neutralization assays revealed the LuS-N71-SpyLinked-CoV-2 spike nanoparticle to elicit potent neutralization responses with geometric mean ID50 titers of 412, 1820, and 1501 for immunization doses of 0.08, 0.4, and 2 μg, respectively (FIG. 6C). In comparison, two doses of soluble trimeric spike elicited neutralization titers at the 0.4 and 2 μg conditions with a geometric mean ID50 of 49 and 315, respectively, with no measurable neutralization at the 0.08 μg dose. In essence, 0.08 μg of spike nanoparticle elicited a neutralization response that was higher, though statistically indistinguishable from 2 μg of trimeric spike. This indicated ˜25-fold higher immunogenicity on a weight-by-weight basis for the spike nanoparticle versus spike alone, suggesting a substantial “dose-sparing” effect. Overall, presentation of the SARS-CoV-2 spike on the LuS nanoparticle surface significantly improved its immunogenicity and required a lower immunogen dose to elicit potent neutralization responses compared with the trimeric form.

It will be apparent that the precise details of the methods or compositions described may be varied or modified without departing from the spirit of the described embodiments. We claim all such modifications and variations that fall within the scope and spirit of the claims below.

1. An immunogen, comprising:
a recombinant SARS-CoV-2 S ectodomain trimer comprising protomers comprising an amino acid sequence at least 95% identical to residues 16-1208 of SEQ ID NO: 2 and comprising proline substitutions at positions 986 and 987 of SEQ ID NO: 2 that stabilize the S ectodomain trimer in a prefusion conformation.

2. The immunogen of claim 1, wherein the protomers in the recombinant SARS-CoV-2 S ectodomain trimer comprise an amino acid sequence at least 98% identical to residues 16-1208 of SEQ ID NO: 2 and comprise the two amino acid substitutions.

3. The immunogen of claim 2, wherein the protomers in the recombinant SARS-CoV-2 S ectodomain trimer comprise an amino acid sequence at least 99% identical to residues 16-1208 of SEQ ID NO: 2 and comprise the two amino acid substitutions.

4. The immunogen of claim 1, wherein the protomers in the recombinant SARS-CoV-2 S ectodomain trimer comprise the amino acid sequence set forth as residues 16-1208 of SEQ ID NO: 2.

5. The immunogen of claim 1, wherein the one or to amino acid substitutions are K986P and V987P substitutions relative to a native SARS-CoV-2 S sequence set forth as SEQ ID NO: 1.

6. The immunogen of claim 1, wherein the protomers of the recombinant SARS-CoV-2 S ectodomain trimer further comprise one or more additional amino acid substitutions that stabilize the recombinant SARS-CoV-2 S ectodomain trimer in the prefusion conformation.

7. The immunogen of claim 1, wherein the protomers in the recombinant SARS-CoV-2 S ectodomain trimer further comprise one or more of N501Y, K417N, and E484K substitutions.

8. The immunogen of claim 1, wherein a C-terminal residue of the protomers in the ectodomain is linked to a trimerization domain by a peptide linker, or is directly linked to the trimerization domain.

9. The immunogen of claim 8, wherein the trimerization domain is a T4 fibritin trimerization domain.

10. The immunogen of claim 8, wherein the protomers linked to the T4 fibritin trimerization domain comprise an amino acid sequence at least 95% identical to residues 16-1235 of SEQ ID NO: 2 and comprise the amino acid substitutions that stabilize the S ectodomain trimer in the prefusion conformation.

11. The immunogen of claim 9, wherein the protomers linked to the T4 fibritin trimerization domain comprise residues 16-1235 of SEQ ID NO: 2.

12. The immunogen of claim 1, wherein a S1/S2 protease cleavage site of the S ectodomain is mutated to inhibit protease cleavage.

13. The immunogen of claim 1, wherein the recombinant SARS-CoV-2 S ectodomain trimer is soluble.

14. The immunogen of claim 1, wherein a C-terminal residue of the protomers in the ectodomain is linked to a transmembrane domain by a peptide linker, or is directly linked to the transmembrane domain.

15. The immunogen of claim 14, wherein the protomers linked to the transmembrane domain comprise an amino acid sequence at least 95% identical to residues 16-1273 of SEQ ID NO: 3 and comprise the amino acid substitutions that stabilize the S ectodomain trimer in the prefusion conformation.

16. The immunogen of claim 14, wherein the protomers linked to the transmembrane domain comprise the amino acid sequence set forth as residues 16-1273 of SEQ ID NO: 3.

17. The immunogen of claim 1, wherein a C-terminal residue of the protomers is linked to a protein nanoparticle subunit by a peptide linker, or is directly linked to the protein nanoparticle subunit.

18. A protein nanoparticle, comprising the immunogen of claim 1.

19. A virus-like particle comprising the immunogen of claim 1.

20. An isolated nucleic acid molecule encoding a protomer of the recombinant SARS-CoV-S ectodomain trimer of claim 1.

21. The nucleic acid molecule of claim 20, operably linked to a promoter.

22. A vector comprising the nucleic acid molecule of claim 20.

23. The vector of claim 22, wherein the vector is a viral vector.

24. An immunogenic composition comprising the immunogen, protein nanoparticle, virus-like particle, nucleic acid molecule, or vector of claim 1, and a pharmaceutically acceptable carrier.

25. A method of producing a recombinant SARS-CoV-2 S ectodomain trimer stabilized in a prefusion conformation, comprising:
expressing the nucleic acid molecule or vector of claim 20 in a host cell to produce the recombinant SARS-CoV-2 S ectodomain trimer; and
purifying the recombinant SARS-CoV-2 S ectodomain trimer.

26. The recombinant SARS-CoV-2 S ectodomain trimer produced by the method of claim 25.

27. A method for generating an immune response to a SARS-CoV-2 S ectodomain in a subject, comprising administering to the subject an effective amount of the immunogen, protein nanoparticle, virus-like particle, nucleic acid molecule, vector, or immunogenic composition of claim 1 to generate the immune response.

28. The method of claim 27, wherein the immune response inhibits infection with SARS-CoV-2.

29. The method of claim 27, wherein generating the immune response inhibits replication of the SARS-CoV-2 in the subject.

30. (canceled)
